(19) United States
(12) Patent Application Publication
(10) Pub. No.: US 2026/0251604 A1
(43) Pub. Date: Aug. 27, 2026

(54) SENSOR DEVICE

(71) Applicant: TDK Corporation, Tokyo (JP)

(72) Inventor: Yutaka MATSUO, Tokyo (JP)

(21) Appl. No.: 19/544,536

(22) Filed: Feb. 19, 2026

(30) Foreign Application Priority Data
Feb. 27, 2025 (JP) 2025-030063

(51) Int. Cl. G01N 27/18; G01N 33/00

(52) U.S. Cl. CPC G01N 27/18; G01N 33/0027

(57) ABSTRACT

Disclosed herein is a sensor device that includes a sensor substrate, a sensor chip mounted on the sensor substrate and having a first sensing element and a second sensing element, and an adhesive member provided between the sensor chip and the sensor substrate so as to fix the sensor chip onto the sensor substrate. The sensor chip has a first region, a second region, and a third region interposed between the first region and the second region in a first direction. The first sensing element is disposed in the first region of the sensor chip. The second sensing element is disposed in the second region of the sensor chip. The adhesive member is disposed predominantly in the third region of the sensor chip.

### CROSS-REFERENCE TO RELATED APPLICATION

This application claims the benefit of Japanese Patent Application No. 2025-030063, filed on February 27, 2025, the entire disclosure of which is incorporated by reference herein.

### BACKGROUND OF THE ART

### Field of the Art

The present disclosure relates to a sensor device and, more particularly, to a sensor device including a sensor chip having a plurality of sensing elements and a sensor substrate on which the sensor chip is mounted.

### Description of Related Art

Japanese Patent No. 7,070,175 discloses a sensor chip having a plurality of sensing elements.

When a sensor device, which includes a sensor substrate on which a sensor chip having a plurality of sensing elements is mounted, is mounted on another product substrate, a large difference in the influence of heat conduction from a heating member mounted on the product substrate may occur between the sensing elements.

### SUMMARY

A sensor device according to an aspect of the present disclosure includes: a sensor substrate; a sensor chip mounted on the sensor substrate and having a first sensing element and a second sensing element; and an adhesive member provided between the sensor chip and the sensor substrate so as to fix the sensor chip onto the sensor substrate, wherein the sensor chip has a first region, a second region, and a third region interposed between the first region and the second region in a first direction, the first sensing element is disposed in the first region of the sensor chip, the second sensing element is disposed in the second region of the sensor chip, and the adhesive member is disposed predominantly in the third region of the sensor chip.

### BRIEF DESCRIPTION OF THE DRAWINGS

The above features and advantages of the present disclosure will be more apparent from the following description of some embodiments taken in conjunction with the accompanying drawings, in which:

FIG. 1 is a schematic plan view illustrating the configuration of a sensor device 100 according to a first embodiment of the technology described herein;

FIG. 2 is a schematic cross-sectional view of the sensor device 100;

FIG. 3 is a circuit diagram of a gas sensor 50 using the sensor device 100;

FIG. 4 is a schematic plan view illustrating the configuration of a sensor device 100A according to a first modification;

FIG. 5 is a schematic plan view illustrating the configuration of a sensor device 100B according to a second modification;

FIG. 6 is a schematic plan view illustrating the configuration of a sensor device 100C according to a third modification;

FIG. 7 is a schematic cross-sectional view of the sensor device 100C;

FIG. 8 is a schematic plan view illustrating the configuration of a sensor device 200 according to a second embodiment of the technology described herein;

FIG. 9 is a schematic plan view illustrating the configuration of the sensor device 200A according to a fourth embodiment;

FIG. 10 is a schematic plan view illustrating the configuration of a sensor device 200B according to a fifth modification;

FIG. 11 is a circuit diagram of the sensor device 200B;

FIG. 12 is a schematic plan view illustrating the configuration of a sensor device 300 according to a third embodiment of the technology described herein;

FIG. 13 is a schematic plan view illustrating the configuration of a sensor device 300A according to a sixth modification;

FIG. 14 is a schematic plan view illustrating the configuration of a sensor device 400 according to a fourth embodiment of the technology described herein;

FIG. 15 is a schematic view for explaining the positional relationship between the sensing elements S1 to S3 and the adhesive member 40;

FIG. 16 is a schematic plan view illustrating the configuration of a sensor device 400A according to a seventh modification;

FIG. 17 is a schematic plan view illustrating the configuration of a sensor device 500 according to a fifth embodiment of the technology described herein; and

FIG. 18 is a schematic plan view illustrating the configuration of a sensor device 600 according to a sixth embodiment of the technology described herein.

### DETAILED DESCRIPTION OF THE EMBODIMENTS

The present disclosure provides a technique for reducing the difference in the influence of heat conduction from a heating member between sensing elements in a sensor device including a sensor chip having sensing elements and a sensor substrate on which the sensor chip is mounted.

Some embodiments of the present disclosure will be explained below in detail with reference to the accompanying drawings.

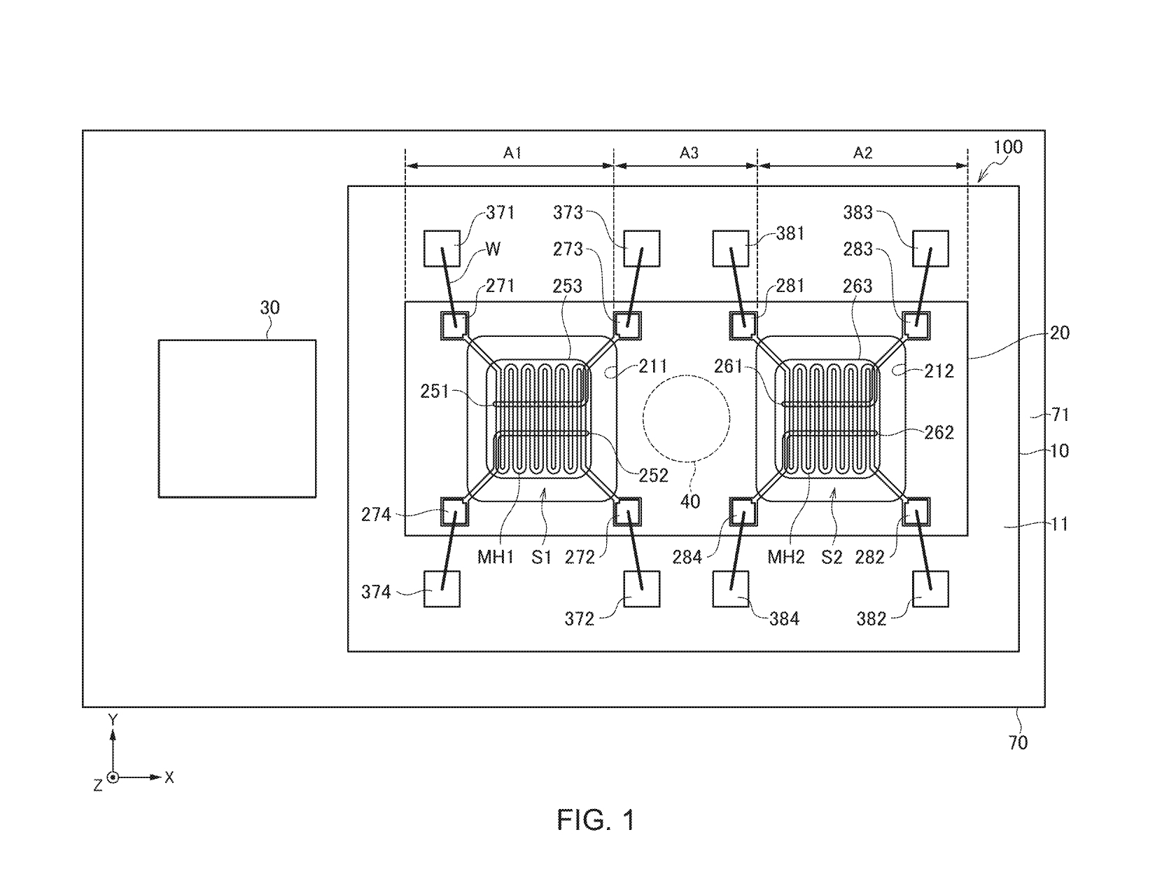

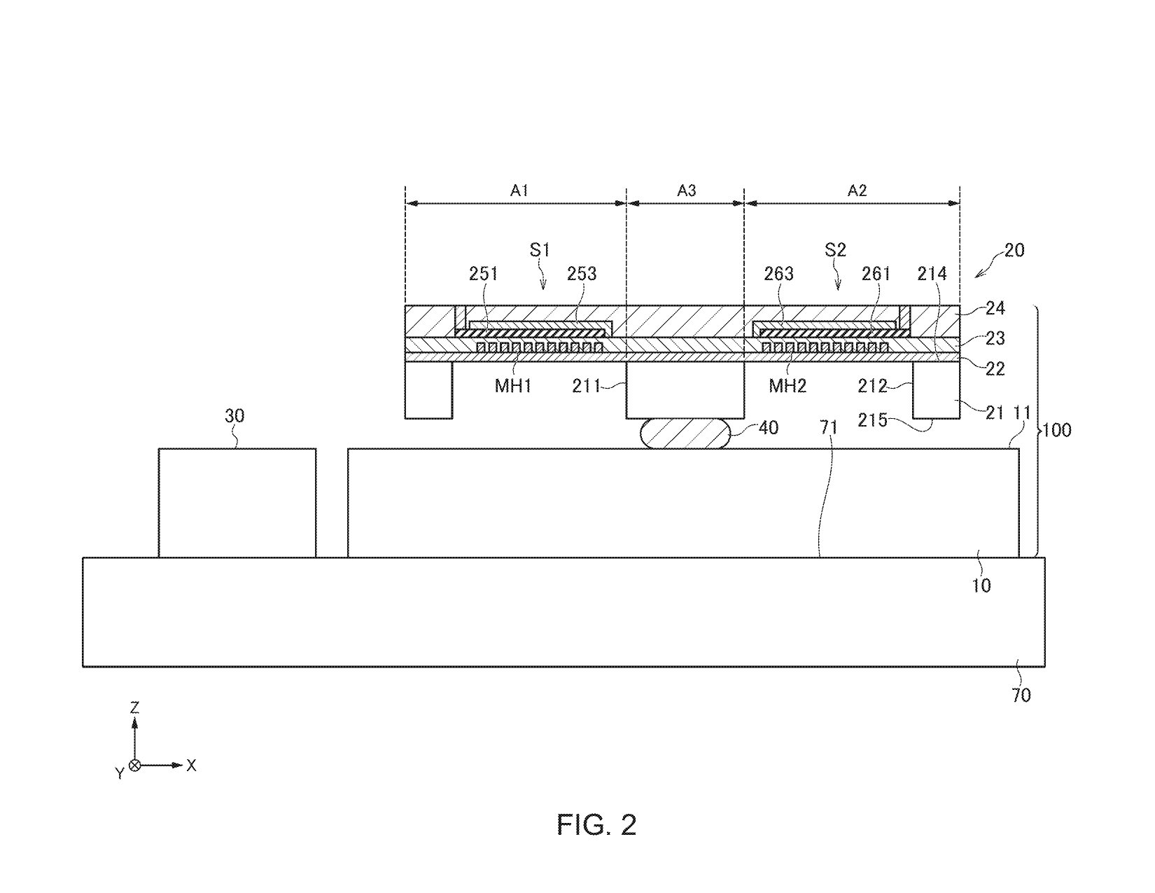

FIG. 1 is a schematic plan view illustrating the configuration of a sensor device 100 according to a first embodiment of the technology described herein. FIG. 2 is a schematic cross-sectional view of the sensor device 100.

As illustrated in FIG. 2, the sensor device 100 according to the first embodiment includes a sensor substrate 10 and a sensor chip 20 mounted on the sensor substrate 10. The sensor substrate 10 has a main surface 11 constituting the XY plane, on which the sensor chip 20 is mounted. The sensor substrate 10 may further have other electronic components mounted thereon. In the example illustrated in FIGS. 1 and 2, the sensor device 100 is mounted on a mounting surface 71 of a product substrate 70. On the mounting surface 71 of the product substrate 70, not only the sensor device 100 but also an electronic component 30 is also mounted. The electronic component 30 is a heating member that generates heat during its operation. In the present embodiment, the electronic component 30 is disposed on the negative side in the X-direction of the sensor device 100 in a plan view as seen from the Z-direction.

The sensor chip 20 has two sensing elements S1 and S2. Although not particularly limited, the sensor chip 20 is a heat conduction type gas sensor chip for detecting, for example, the concentration of CO₂ gas in measuring atmosphere. The sensor chip 20 outputs a signal based on the state of the sensing element S1 and the state of the sensing element S2. The signal that the sensor chip 20 outputs is, for example, a signal indicating the concentration of CO₂ gas in a measuring atmosphere. In the present embodiment, the sensing element S1 is a temperature-sensitive element for detection, and the sensing element S2 is a temperature-sensitive element for reference. The sensor chip 20 includes: a substrate 21 having a thickness in the Z-direction; an insulating film 22 covering a surface 214 of the substrate 21 on the positive side in the Z-direction; heaters MH1 and MH2 provided on the insulating film 22; an insulating film 23 covering the heaters MH1 and MH2; a pair of thermistor electrodes 251 and 252 provided on the insulating film 23 so as to overlap the heater MH1 in a plan view as seen from the Z-direction; a pair of thermistor electrodes 261 and 262 provided on the insulating film 23 so as to overlap the heater MH2 in a plan view as seen from the Z-direction; a thermistor resistor 253 covering the pair of thermistor electrodes 251 and 252; a thermistor resistor 263 covering the pair of thermistor electrodes 261 and 262; an insulating film 24 covering the thermistor electrodes 251, 252, 261, and 262 and thermistor resistors 253 and 263; and a plurality of pad electrodes 271 to 274 and 281 to 284 provided on the insulating film 24.

The pair of thermistor electrodes 251, 252 and thermistor resistor 253 constitute the sensing element S1. The pair of thermistor electrodes 261, 262 and thermistor resistor 263 constitute the sensing element S2. The sensing elements S1 and S2 are arranged in the X-direction (first direction). As seen from the center of the sensor chip 20, the sensing element S1 is located on the negative side in the X-direction, and the sensing element S2 is located on the positive side in the X-direction. The sensing element S1 is located closer to the electronic component 30 than the sensing element S2.

The material of the substrate 21 is not particularly limited as long as it has adequate mechanical strength and can be suitably subjected to fine processing such as etching, and examples of the substrate 21 include a silicon substrate, a sapphire substrate, a ceramic substrate, a quartz substrate, and a glass substrate. The substrate 21 has cavities 211 and 212 at positions overlapping the heaters MH1 and MH2, respectively, in a plan view as seen from the Z-direction in order to enhance thermal efficiency of the heaters MH1 and MH2. In the region where the cavity 211 is formed, the substrate 21 is locally reduced in thickness or removed. In the example illustrated in FIGS. 1 and 2, the substrate 21 is removed in the cavities 211 and 222, and the heaters MH1, MH2 and the thermistor resistors 253, 263 are supported by the insulating film 22. The insulating film 22 in a region that does not overlap any of the heaters MH1, MH2 and the thermistor resistors 253, 263 in a plan view may be removed.

The insulating films 22 to 24 may be made of an inorganic insulating material such as silicon oxide or silicon nitride. The heaters MH1 and MH2 each a meandered wire structure formed of a metal material with a relatively high melting point, such as molybdenum (Mo), platinum (Pt), gold (Au), tungsten (W), tantalum (Ta), palladium (Pd), iridium (Ir), or an alloy containing two or more of these metals. One end of the heater MH1 is connected to the pad electrode 271 and the other end thereof is connected to the pad electrode 272. One end of the heater MH2 is connected to the pad electrode 281 and the other end thereof is connected to the pad electrode 282.

The thermistor resistors 253 and 263 is made of a material whose resistance varies with temperature, such as vanadium oxide, amorphous silicon, polycrystalline silicon, an oxide with a spinel crystal structure containing manganese, titanium oxide, or yttrium-barium-copper oxide. For example, the thermistor resistors 253 and 263 are NTC thermistors having a negative temperature coefficient of resistance. The pair of thermistor electrodes 251 and 252 are in contact with the thermistor resistor 253. Thus, the resistance between the thermistor electrodes 251 and 252 is defined by the resistance of the thermistor resistor 253 located between the electrodes. The thermistor electrodes 251 and 252 are connected to the pad electrodes 273 and 274, respectively. Similarly, the pair of thermistor electrodes 261 and 262 are in contact with the thermistor resistor 263. Thus, the resistance between the thermistor electrodes 261 and 262 is defined by the resistance of the thermistor resistor 263 located between the electrodes. The thermistor electrodes 261 and 262 are connected to the pad electrodes 283 and 284, respectively.

As illustrated in FIGS. 1 and 2, the sensor chip 20 is fixed to the sensor substrate 10 by an adhesive member 40. The adhesive member 40 is provided between a back surface 215 (surface of the substrate 21 on the negative side in the Z-direction) of the substrate 21 constituting the sensor chip 20 and the main surface 11 of the sensor substrate 10.

The sensor chip 20 has a region A1 located on the negative side in the X-direction, a region A2 located on the positive side in the X-direction, and a region A3 interposed between the regions A1 and A2 in the X-direction. The region A3 has a fixed width in the X-direction. The regions A1 and A3 are contiguous, and the boundary therebetween extends in the Y-direction (second direction) perpendicular to the X-direction in a plan view as seen from the Z-direction. The regions A2 and A3 are contiguous, and the boundary therebetween extends in the Y-direction (second direction) perpendicular to the X-direction in a plan view as seen from the Z-direction. The sensing element S1 is located in the region A1, the sensing element S2 is located in the region A2, and the adhesive member 40 is located in the region A3. In the present embodiment, the adhesive member 40 is not provided in the regions A1 and A2.

The pad electrodes 271 to 274 and 281 to 284 provided on the sensor chip 20 are connected to pad electrodes 371 to 374 and 381 to 384 provided on the main surface 11 of the sensor substrate 10, respectively, through the bonding wires W. In the example illustrated in FIG. 1, the pad electrodes 371, 373, 381, and 383 are disposed on the positive side in the Y-direction as seen from the sensor chip 20, and the pad electrodes 372, 374, 382, and 384 are disposed on the negative side in the Y-direction as seen from the sensor chip 20.

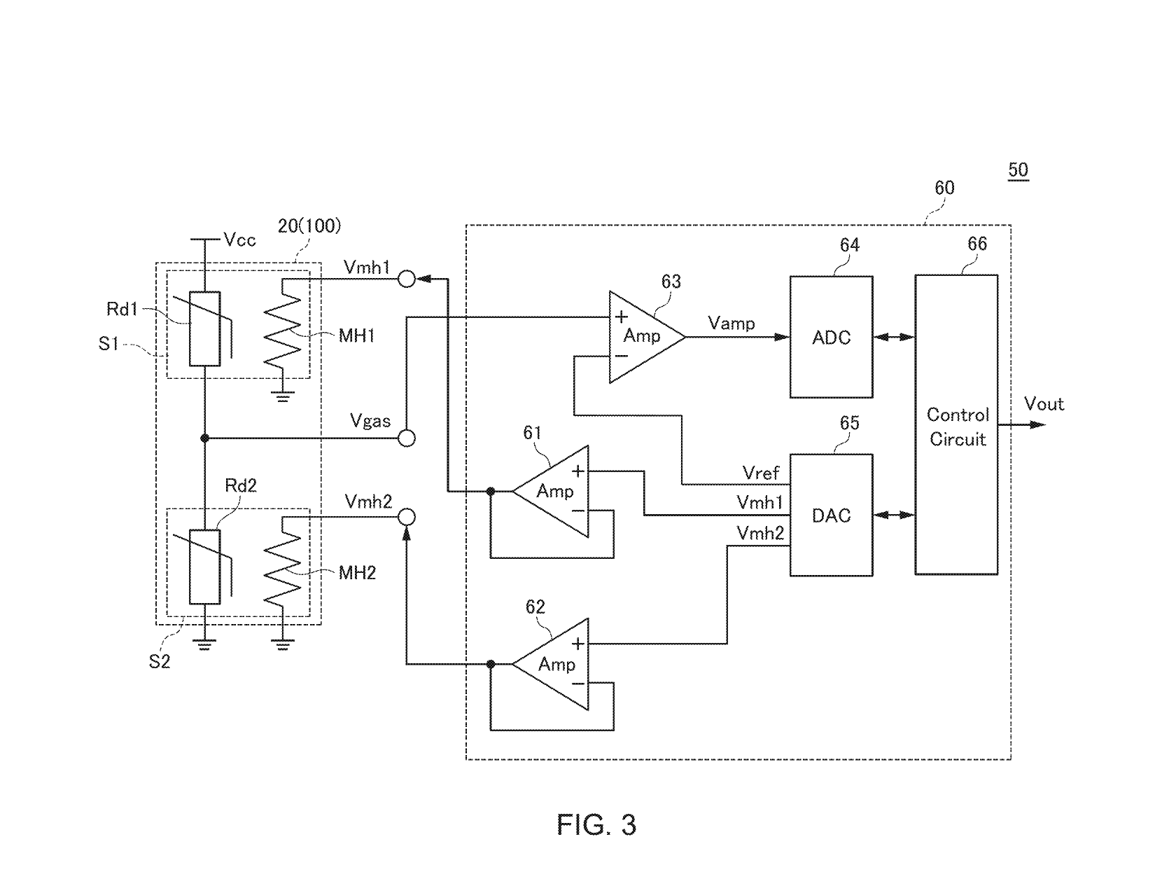

FIG. 3 is a circuit diagram of a gas sensor 50 using the sensor device 100.

The gas sensor 50 illustrated in FIG. 3 is composed of the sensor chip 20 included in the sensor device 100 and a signal processing circuit 60 connected to the sensor chip 20. The sensor chip 20 includes thermistors Rd1 and Rd2 connected in series in this order between a power supply Vcc and a ground GND and the heaters MH1 and MH2. The thermistor Rd1 is composed of the thermistor resistor 253 and the pair of thermistor electrodes 251 and 252, all of which are illustrated in FIG. 1. The thermistor Rd2 is composed of the thermistor resistor 263 and the pair of thermistor electrodes 261 and 262, all of which are illustrated in FIG. 1.

The thermistor Rd1 varies in temperature in response to a change in the temperature of the heater MH1. The thermistor Rd2 varies in temperature in response to a change in the temperature of the heater MH2. A gas detection signal Vgas appears at the node between the thermistors Rd1 and Rd2. The thermistor Rd1 is a temperature-sensitive element for detection, and the thermistor Rd2 is a temperature-sensitive element for reference.

In measuring a gas concentration using the gas sensor 50, the thermistor Rd1 is heated to around 150°C (an example of a first temperature range) by the heater MH1, while the thermistor Rd2 is heated to around 300°C (an example of a second temperature range) by the heater MH2. The first temperature range is a predetermined temperature range included within a range of 100°C or more and 230°C or less, for example, a temperature range around 150°C. The second temperature range is a predetermined temperature range included within a range of 250°C or more and 450°C or less, for example, a temperature range around 300°C. The term “temperature range” in the present specification refers to a range having, for example, a width of 1°C or less. Thus, for example, the temperature range around 150°C may be from 149.5°C to 150.5°C, for example. Further, for example, the temperature range around 300°C may be from 299.5°C to 300.5°C, for example. The thermistor Rd1 is designed to have a predetermined resistance value when heated to 150°C, while the thermistor Rd2 is designed to have a predetermined resistance value when heated to 300°C. The first temperature range (around 150°C in this example) and the second temperature range (around 300°C in this example) differ from each other have different temperature ranges, and in this example, the first temperature range is lower than the second temperature range.

When CO₂ gas is present in the measurement atmosphere in a state in which the thermistor Rd1 as the temperature-sensitive element for detection is heated to around 150°C, the heat dissipation characteristics of the thermistor Rd1 change in accordance with the concentration of CO₂ gas. This change appears as a change in the temperature of the thermistor Rd1, that is, a change in the resistance thereof. For example, in the temperature range around 150°C, the thermal conductivity of CO₂ gas is lower than that of air, so that the temperature of the thermistor Rd1 rises as the concentration of CO₂ gas increases. Therefore, when the thermistor Rd1 is heated such that its temperature reaches 150°C in a measuring atmosphere in which the CO₂ gas concentration is zero, if CO₂ gas is present in the measurement atmosphere, the temperature of the thermistor Rd1 may increase above 150°C depending on the gas concentration. As a result, the resistance of the thermistor Rd1 decreases as the CO₂ gas concentration in the measurement atmosphere increases.

On the other hand, even when CO₂ gas is present in the measurement atmosphere in a state where the thermistor Rd2 as the temperature-sensitive element for reference is heated to around 300°C, the heat dissipation characteristics of the thermistor Rd2 hardly changes depending on the CO₂ concertation, and its temperature also hardly changes. Accordingly, a change in the resistance of the thermistor Rd2 heated to around 300°C depending on the concentration of CO₂ gas is sufficiently smaller than that of the thermistor Rd1 heated to around 150°C depending on the concentration of CO₂ gas. The change in the resistance of the thermistor Rd2 heated to around 300°C depending on the CO₂ gas concentration may be negligible. As a result, when the thermistors Rd1 and Rd2 are heated to around 150°C and around 300°C, respectively, (when the thermistors Rd1 and Rd2 are heated such that their temperatures reach 150°C and 300°C, respectively, in a measuring atmosphere in which the CO₂ gas concentration is zero), the gas detection signal Vgas corresponding to the CO₂ gas concentration in the measurement atmosphere appears at the node between the thermistors Rd1 and Rd2. On the other hand, even when another gas whose heat dissipation characteristics exhibit no significant difference between when the thermistor Rd1 is heated to around 150°C and when the thermistor Rd1 is heated to around 300°C is contained in the measurement atmosphere, the concentration of this gas has little influence on the level of the gas detection signal Vgas. This allows the sensor chip 20 to selectively detect the concentration of CO₂ gas.

The signal processing circuit 60 includes differential amplifiers 61 to 63, an AD converter (ADC) 64, a DA converter (DAC) 65, and a control circuit 66. The signal processing circuit 60 may be provided, wholly or partially, on the sensor substrate 10. Alternatively, the signal processing circuit 60 may be provided, wholly or partially, on a substrate (e.g., product substrate 70) other than the sensor substrate 10.

The differential amplifier 63 is configured to compare the gas detection signal Vgas with a reference potential Vref output from the DA converter 65 to generate an amplification signal Vamp corresponding to the amplified level difference (= Vgas – Vref) between the gas detection signal Vgas and the reference potential Vref. The amplification signal Vamp is input to the AD converter 64. The AD converter converts the amplification signal Vamp into its corresponding digital value and supplies it to the control circuit 66.

The control circuit 66 calculates the concentration of CO₂ gas, which is a gas to be measured, based on the AD-converted amplification signal Vamp1 and generates an output signal Vout indicating the CO₂ gas concentration. The output signal Vout is output outside the gas sensor 50. The control circuit 66 may calculate the CO₂ gas concentration using a calculation formula set therein. Further, the control circuit 66 supplies digital values of various control parameters to the DA converter 65. The DA converter 65 DA-converts the digital values of the various control parameters to generate heater voltages Vmh1 and Vmh2 and the reference potential Vref. The heater voltage Vmh1 is applied to the heater MH1 through the differential amplifier 61 constituting a voltage follower to heat the heater MH1. The heater voltage Vmh2 is applied to the heater MH2 through the differential amplifier 62 constituting a voltage follower to heat the heater MH2.

As illustrated in FIGS. 1 and 2, the sensor chip 20 is fixed to the sensor substrate 10 through the adhesive member 40 provided in the region A3. If the adhesive members 40 are disposed in the regions A1 and A2 of the sensor chip 20, for example, heat conducted from the electronic component 30 to the sensor chip 20 through the product substrate 70 and sensor substrate 10 is greater at the sensing element S1 than at the sensing element S2 due to the dominance of the heat component conducted through the adhesive member 40 disposed in the region A, causing a large difference between the amount of heat conducted to the sensing element S1 through the product substrate 70, the sensor substrate 10, and the adhesive member 40 and that conducted to the sensing element S2 through the product substrate 70, the sensor substrate 10, and the adhesive member 40. That is, the thermistor Rd1 as the temperature-sensitive element for detection is subjected to a greater amount of heat from the electronic component 30 than the thermistor Rd2 as the temperature-sensitive element for reference. As described using FIG. 3, the thermistors Rd1 and Rd2 constitute a half-bridge circuit, so that the large difference between the amount of heat conducted to the sensing element S1 through the product substrate 70, the sensor substrate 10, and the adhesive member 40 and that conducted to the sensing element S2 through the product substrate 70, the sensor substrate 10, and the adhesive member 40 may result in a large measurement error.

On the other hand, in the present embodiment, the adhesive member 40 is disposed in the region A3 located between the region A1 where the sensing element S1 is disposed and the region A2 where the sensing element S2 is disposed. In other words, the sensing elements S1 and S2 are arranged in the X-direction, and in the X-direction in a plan view as seen from the Z-direction, the X-direction position of the adhesive member 40 disposed in the region A3 is between the X-direction positions of the sensing elements S1 and S2. Thus, even when the electronic component 30 generates heat during its operation, it is possible to reduce the difference between the amount of heat conducted from the electronic component 30 to the sensing element S1 through the product substrate 70, the sensor substrate 10, and the adhesive member 40 and that conducted from the electronic component 30 to the sensing element S2 through the product substrate 70, the sensor substrate 10, and the adhesive member 40. That is, the difference in the influence of heat conduction from the electronic component 30 between the sensing elements S1 and S2 can be reduced, making it possible to reduce a measurement error caused by the heat generation of the electronic component 30.

Further, in the present embodiment, the adhesive member 40 is interposed between the sensing elements S1 and S2 in the X-direction in a plan view as seen from the Z-direction (thickness direction of the sensor substrate 10), making it possible to reduce the size of the sensor chip 20 in the Y-direction crossing the X-direction.

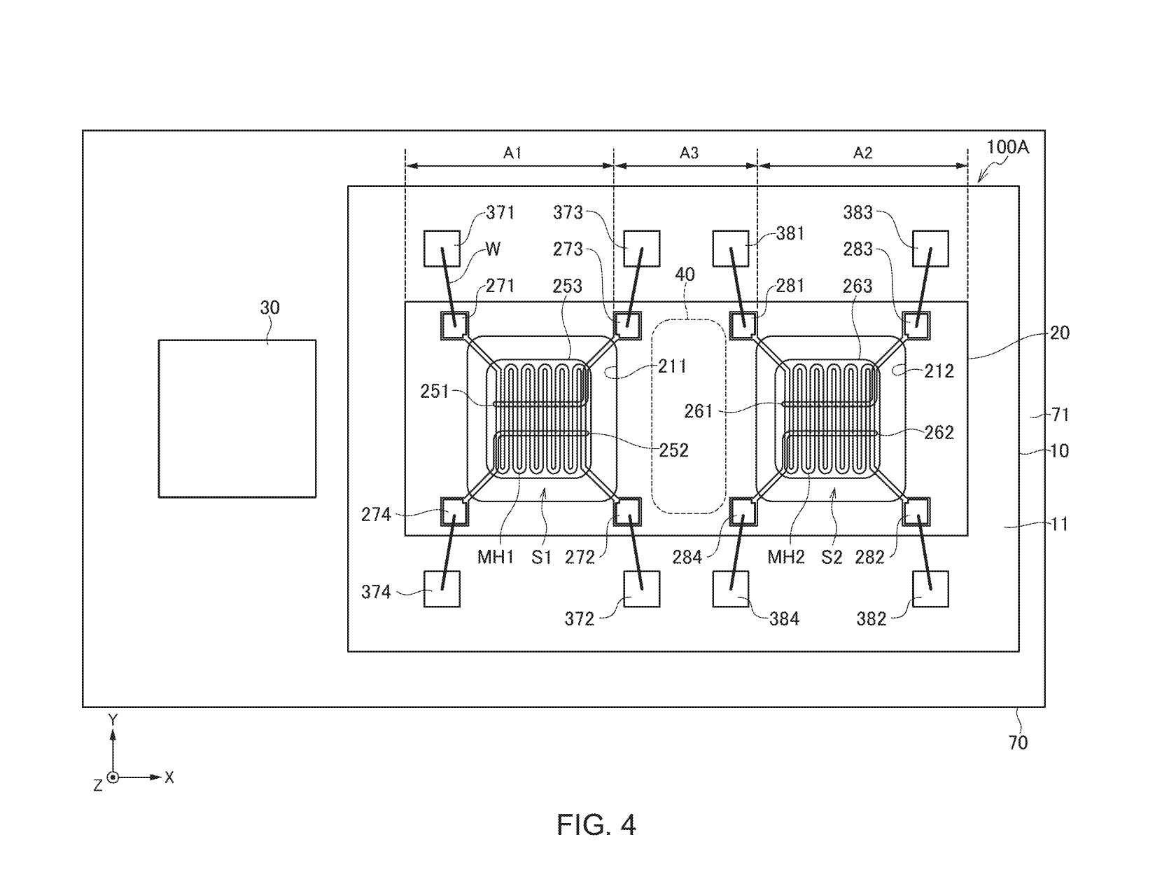

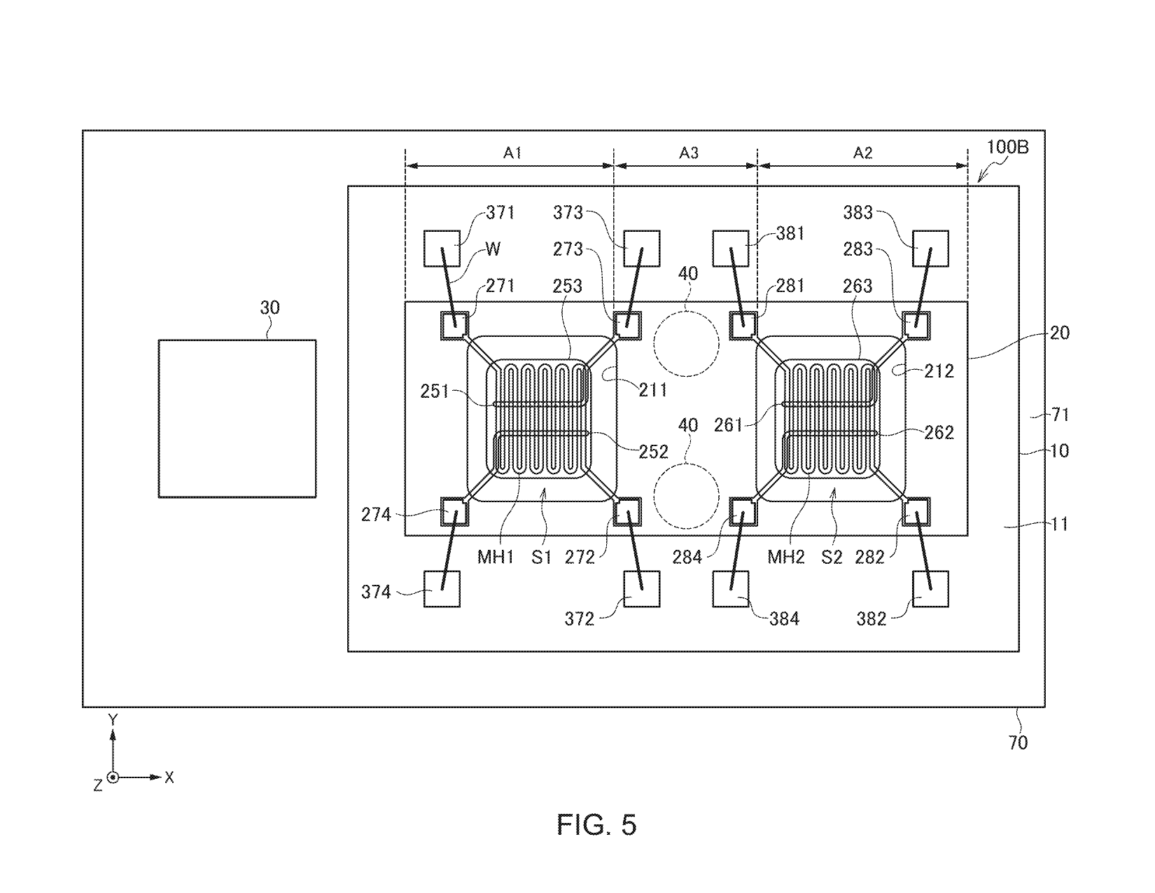

In the example illustrated in FIGS. 1 and 2, the adhesive member 40 has a circular shape when viewed from the Z-direction; however, like a sensor device 100A according to a first modification illustrated in FIG. 4, the adhesive member 40 may have a shape elongated in the Y-direction when viewed from the Z-direction. This allows the sensor chip 20 to be fixed stably on the main surface 11 of the sensor substrate 10. Further, in the example illustrated in FIGS. 1 and 2, the adhesive member 40 is disposed at one location; however, like a sensor device 100B according to a second modification illustrated in FIG. 5, the adhesive member 40 may be disposed at separate locations in the region A3. This allows the sensor chip 20 to be fixed more stably on the main surface 11 of the sensor substrate 10 while reducing the amount of the adhesive member 40. In the first and second modifications, a part of the adhesive member 40 is disposed in the third region A3, interposed between the sensing elements S1 and S2 in the X-direction, and the remaining part is disposed outside the region interposed between the sensing elements S1 and S2 in the X-direction. In the first and second modifications as well, in a plan view as seen from the Z-direction, the X-direction position of the adhesive member 40 disposed in the region A3 is between the X-direction positions of the sensing elements S1 and S2.

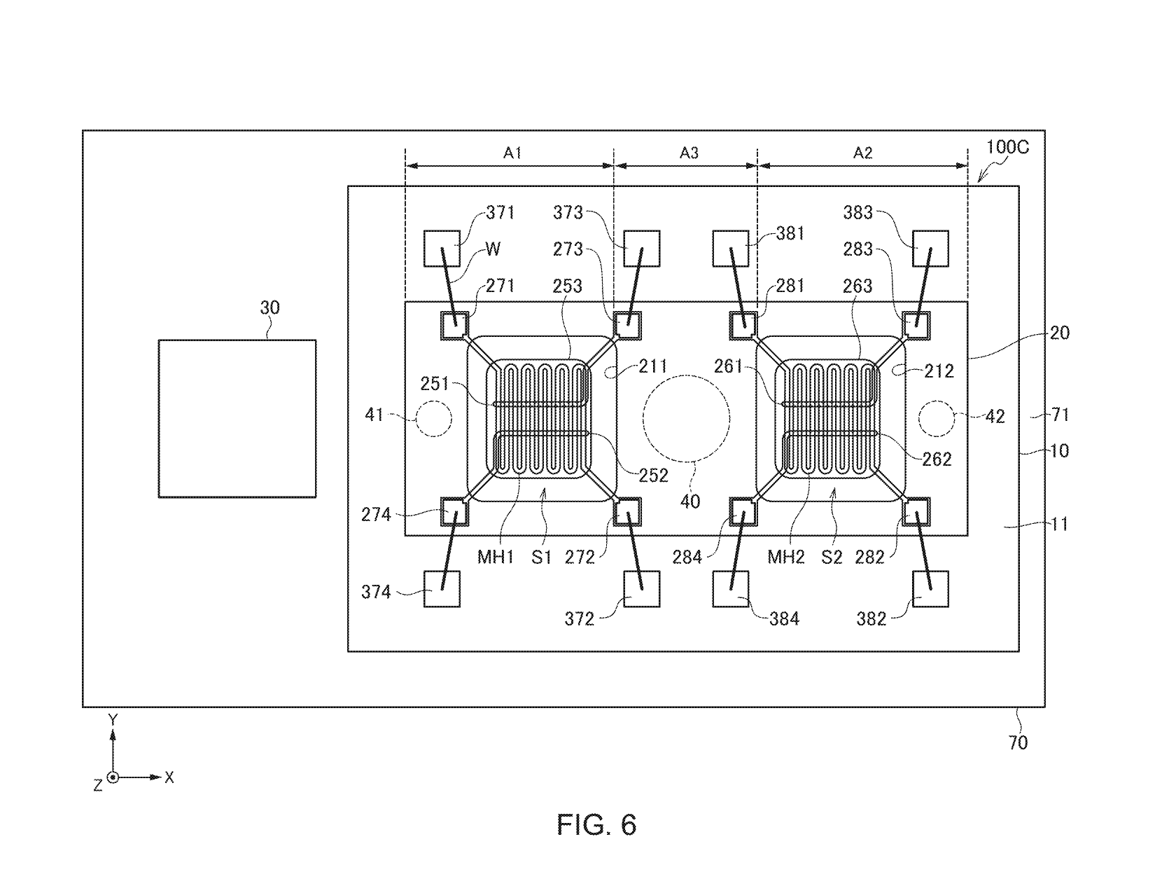

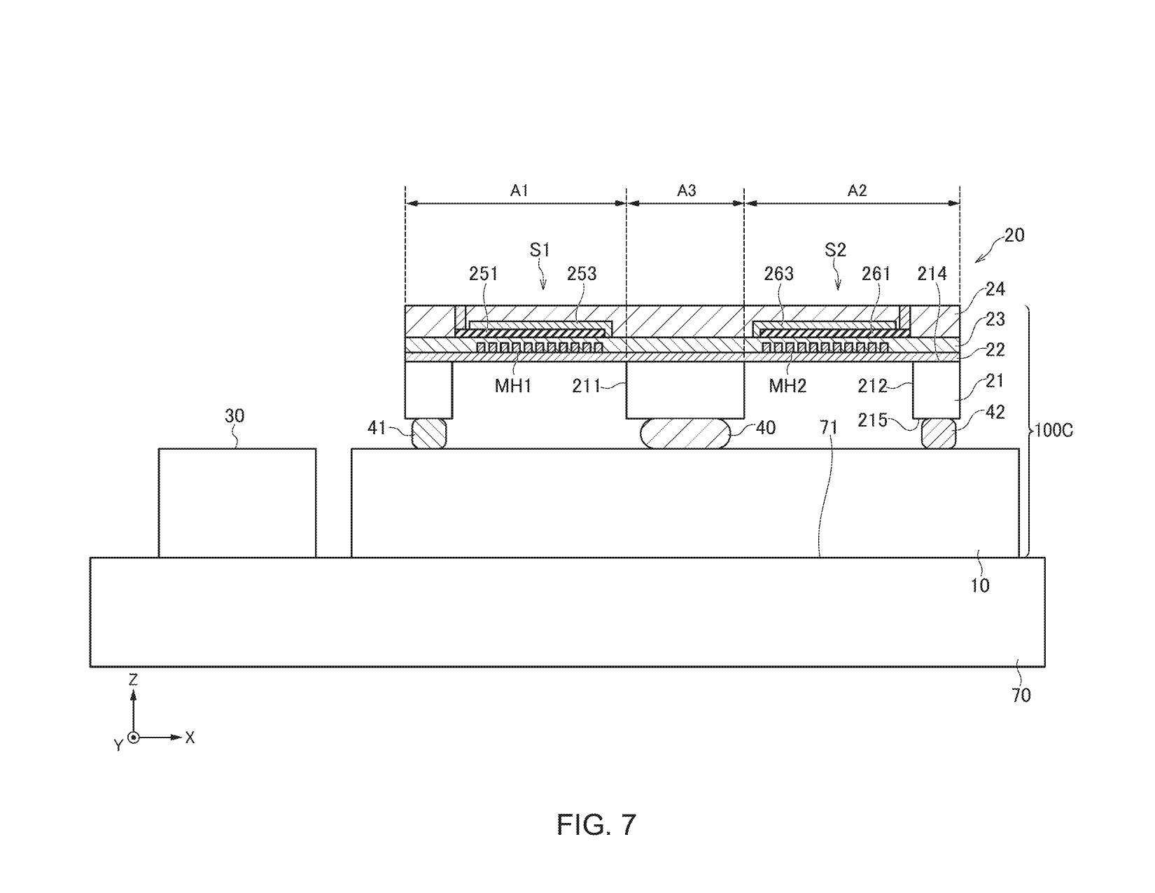

FIG. 6 is a schematic plan view illustrating the configuration of a sensor device 100C according to a third modification. FIG. 7 is a schematic cross-sectional view of the sensor device 100C.

The sensor device 100C according to the third modification illustrated in FIGS. 6 and 7 differs from the sensor device 100 illustrated in FIGS. 1 and 2 in that adhesive members 41 and 42 are additionally provided in the regions A1 and A2 of the sensor chip 20, respectively. The adhesive members 41 and 42 each have a smaller volume than the adhesive member 40 provided in the region A3, and accordingly, the heat conduction from the sensor substrate 10 to the sensor chip 20 is greater through the adhesive member 40 than through each of the adhesive members 41 and 42. That is, the heat conduction from the sensor substrate 10 to the sensor chip 20 is greatest through the adhesive member 40.

As exemplified by the sensor device 100C according to the third modification, even when the adhesive members 41 and 42 are additionally disposed in the regions A1 and A2 of the sensor chip 20, respectively, by disposing the adhesive member 40 predominantly in the region A3 of the sensor chip 20 so that the heat conduction through the adhesive member 40 becomes dominant, it is possible to reduce a measurement error caused by the heat generation of the electronic component 30. In other words, when the adhesive member is predominantly disposed between the X-direction positions of the sensing elements S1 and S2 in a plan view as seen from the Z-direction, it is possible to reduce a measurement error caused by the heat generation of the electronic component 30.

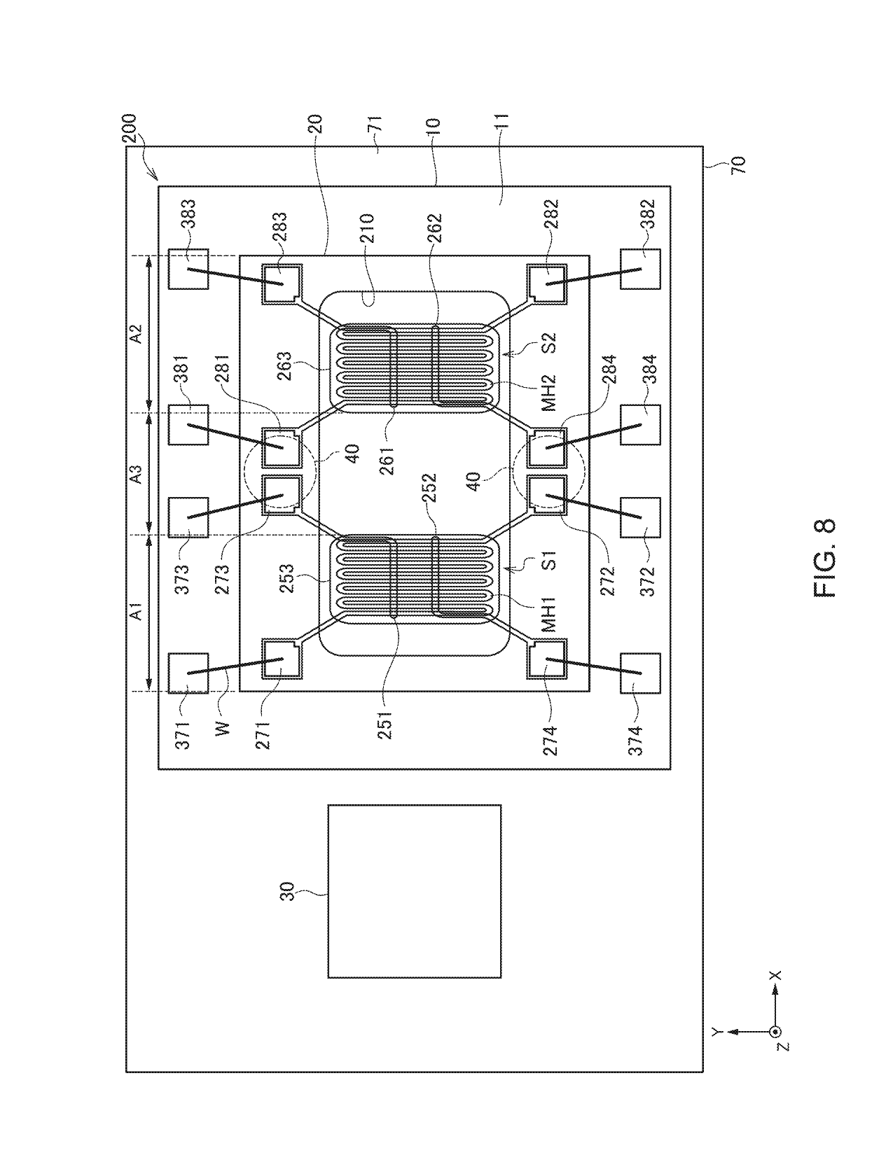

FIG. 8 is a schematic plan view illustrating the configuration of a sensor device 200 according to a second embodiment of the technology described herein.

As illustrated in FIG. 8, the sensor device 200 according to the second embodiment differs from the sensor device 100 according to the first embodiment in that both the sensing elements S1 and S2 are disposed in a single cavity 210 formed in the substrate 21 of the sensor chip 20. Other basic configurations are the same as those of the sensor device 100 according to the first embodiment, so the same reference numerals are given to the same elements, and overlapping description will be omitted.

In the sensor device 200 according to the second embodiment, the adhesive member 40 is disposed at two locations in the region A3 so as to avoid the cavity 210. Thus, in a plan view as seen from the Z-direction, the adhesive member 40 is disposed in the third region A3, outside the region interposed between the sensing elements S1 and S2 in the X-direction. In other words, in a plan view as seen from the Z-direction, the two adhesive members 40 are arranged so as to sandwich the cavity 210 in the Y-direction.

As exemplified by the sensor device 200 according to the second embodiment, the adhesive member 40 need not be interposed between the sensing elements S1 and S2 in the X-direction in a plan view as seen from the Z-direction and may be disposed outside the region interposed between the sensing elements S1 and S2 in the X-direction. Even in this case, it is possible to reduce the difference between the amount of heat conducted from the electronic component 30 to the sensing element S1 through the product substrate 70, the sensor substrate 10, and the adhesive member 40 and that conducted from the electronic component 30 to the sensing element S2 through the product substrate 70, the sensor substrate 10, and the adhesive member 40. That is, the difference in the influence of heat conduction from the electronic component 30 between the sensing elements S1 and S2 can be reduced.

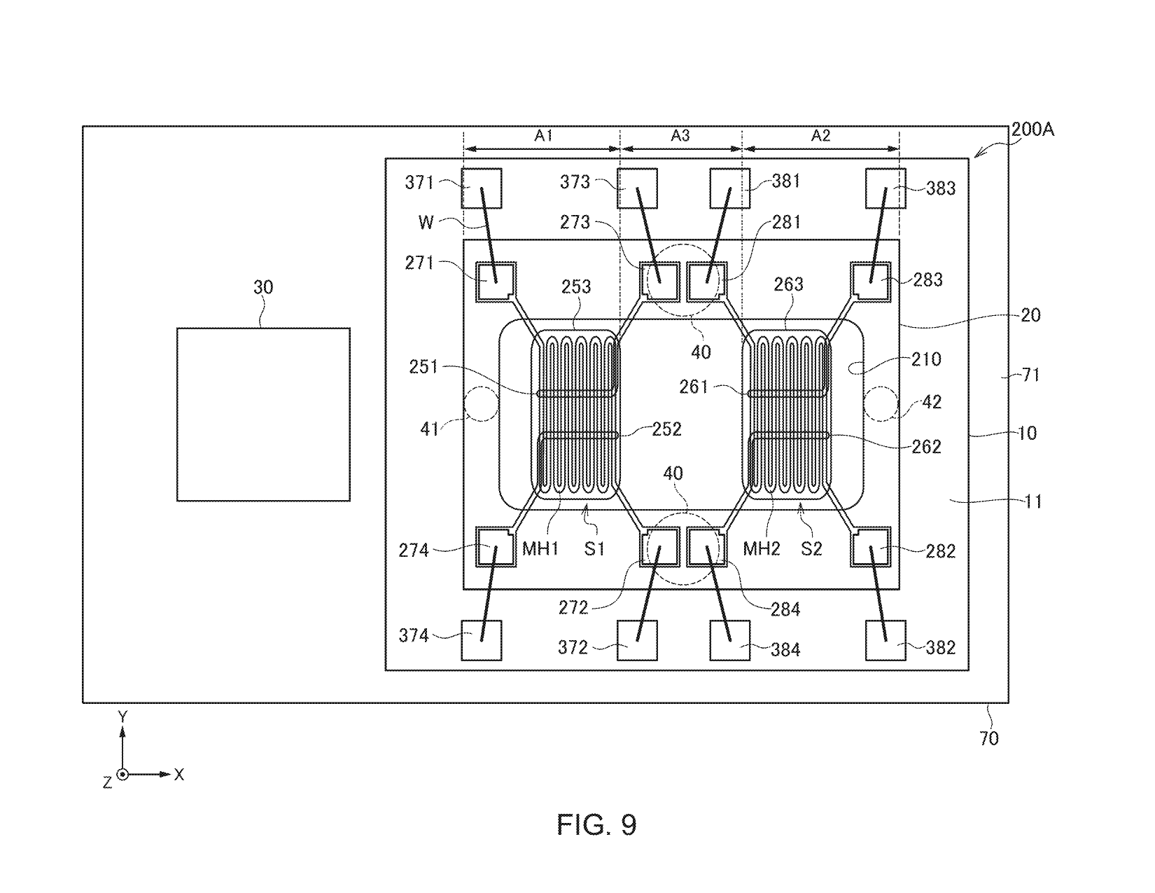

FIG. 9 is a schematic plan view illustrating the configuration of the sensor device 200A according to a fourth modification.

The sensor device 200A according to the fourth modification illustrated in FIG. 9 differs from the sensor device 200 illustrated in FIG. 8 in that the adhesive members 41 and 42 are additionally provided in the regions A1 and A2 of the sensor chip 20, respectively. The adhesive members 41 and 42 each have a smaller volume than the adhesive member 40 provided in the region A3, and the adhesive member is disposed predominantly in the region A3 of the sensor chip. Thus, the heat conduction from the sensor substrate 10 to the sensor chip 20 is greater through the adhesive members 40 than through the adhesive members 41 and 42.

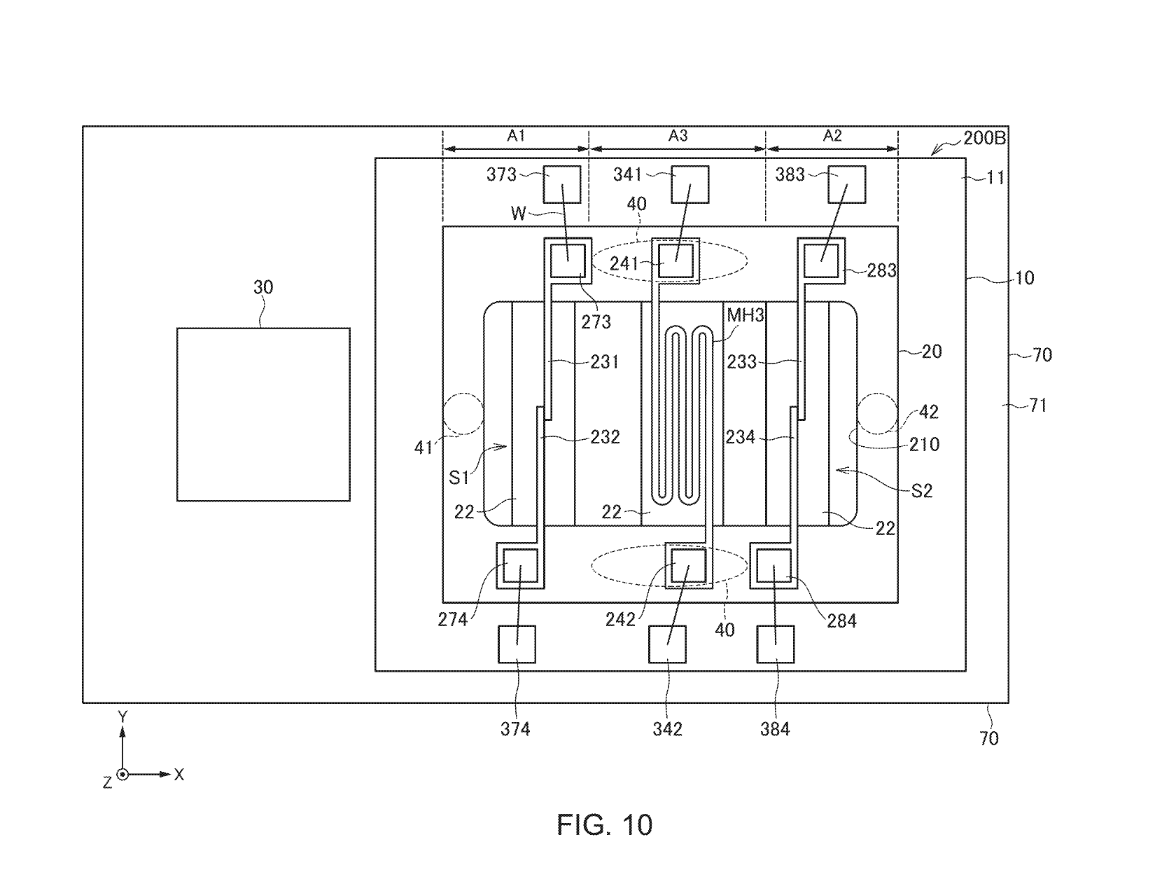

FIG. 10 is a schematic plan view illustrating the configuration of a sensor device 200B according to a fifth modification.

The sensor device 200B according to the fifth modification illustrated in FIG. 10 differs from the sensor device 200A illustrated in FIG. 9 in the following respects; the heaters MH1 and MH2 are removed, and instead a heater MH3 is disposed between the sensing elements S1 and S2; the sensing element S1 is constituted by a thermocouple including metallic conductors 231 and 232; and the sensing element S2 is constituted by a thermocouple including metallic conductors 233 and 234. The metallic conductors 231 to 234 and the heater MH3 may be supported by the insulating film 22. A part of the insulating film 22 located between the sensing element S1 and heater MH3 is removed, and a part of the insulating film 22 located between the sensing element S2 and heater MH3 is removed, with the result that spaces are formed between the sensing element S1 and the heater MH3 and between the sensing element S2 and the heater MH3. One end of the heater MH3 is connected to a pad electrode 241, and the other end thereof is connected to a pad electrode 242. The pad electrodes 241 and242 are connected to pad electrodes 341 and 342, respectively, provided on the main surface 11 of the sensor substrate 10 through the bonding wires W.

The heater MH3 is a common heater for the sensing elements S1 and S2. When the heater MH3 is heated, heat therefrom is conducted to the sensing elements S1 and S2 through the spaces between the heater MH3 and the sensing elements. Here, the distance between the sensing element S1 and the heater MH3 in the X-direction and that between the sensing element S2 and the heater MH3 differ from each other, so that when the heater MH3 is heated, a temperature difference occurs between the sensing elements S1 and S2.

When the concentration of CO₂ gas in the measuring atmosphere varies, the thermal conductivities of the spaces between the heater MH3 and the sensing elements S1 and S2 change, with the result that the temperature difference between the sensing elements S1 and S2 changes. For example, when the concentration of CO₂ gas increases, the thermal conductivities between the heater MH3 and the sensing elements S1 and S2 decrease, so that the temperature difference between the sensing elements S1 and S2 decreases. Although the sensing elements S1 and S2 are each constituted by a single thermocouple in the example of FIG. 10, they may each be constituted by a plurality of thermocouples. That is, the sensing elements S1 and S2 may each be a thermopile element.

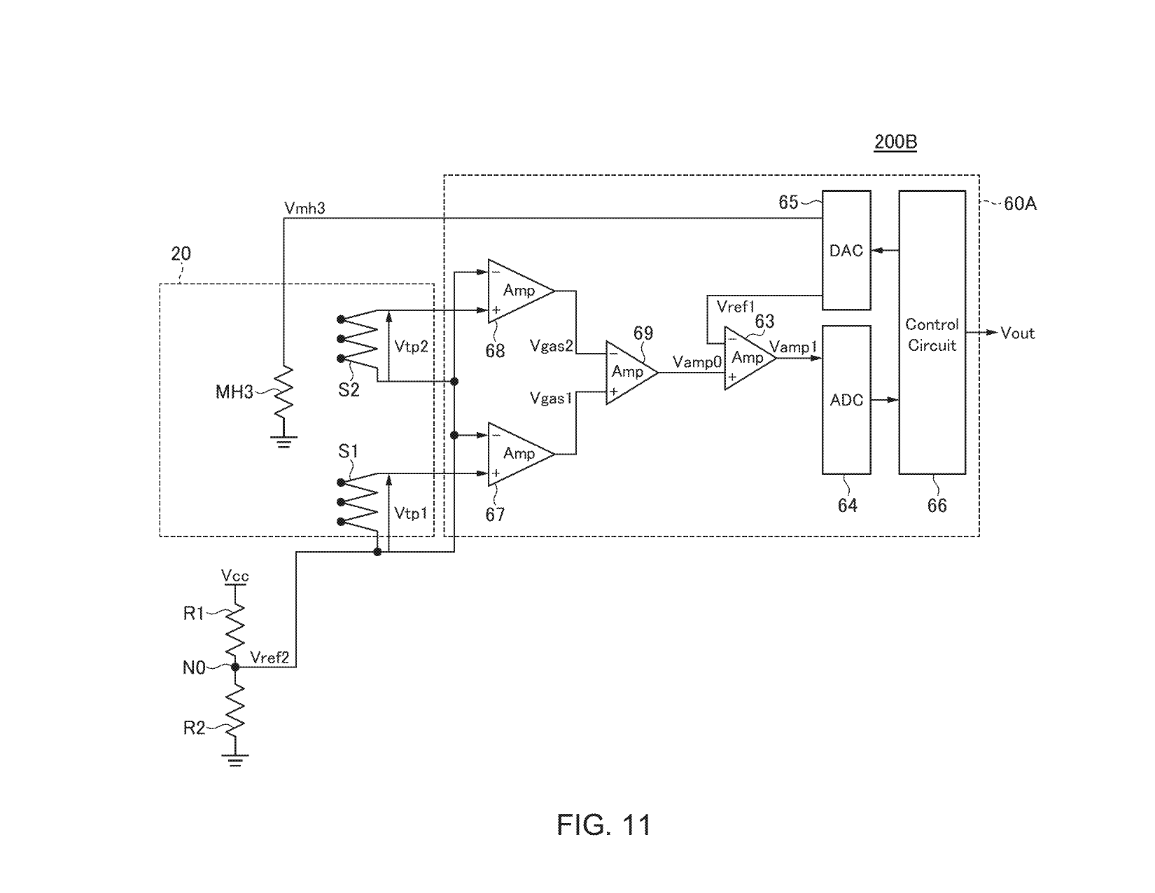

FIG. 11 is a circuit diagram of the sensor device 200B.

As illustrated in FIG. 11, the sensor device 200B is composed of the sensor chip 20 illustrated in FIG. 10 and a signal processing circuit 60A connected to the sensor chip 20. The sensor chip 20 includes the sensing elements S1 and S2, which are thermocouples or thermopile elements, and the heater MH3 for heating the sensing elements S1 and S2. When the heater MH3 is heated, a heater voltage Vmh3 supplied from the DA converter 65 is applied to the heater MH3.

The hot junctions of the sensing elements S1 and S2 change in temperature in response to a change in temperature of the heater MH3. The sensing elements S1 and S2 are each a temperature-sensitive element in which a potential difference between both ends thereof varies depending on temperature. The potential difference between both ends of the sensing element S1 is used as an output signal Vtp1, and the potential difference between both ends of the sensing element S2 is used as an output signal Vtp2. A reference potential Vref2 is generated by fixed resistors R1 and R2. The fixed resistors R1 and R2 are connected in series between the power supply Vcc and the ground GND, and the reference potential Vref2 appears at the node N0 therebetween. The reference potential Vref2 is supplied in common to the inversion input terminals (-) of differential amplifiers 67 and 68 included in the signal processing circuit 60A. The output signal Vtp1 is supplied to the non-inversion input terminal (+) of the differential amplifier 67 included in the signal processing circuit 60A, and the output signal Vtp2 is supplied to the non-inversion input terminal (+) of the differential amplifier 68 included in the signal processing circuit 60A.

The potential supplied to the non-inversion input terminal (+) of the differential amplifier 67 has a level obtained by superimposing the output signal Vtp1 corresponding to the temperature-dependent electromotive force of the sensing element S1 on the reference potential Vref2. The potential supplied to the non-inversion input terminal (+) of the differential amplifier 68 has a level obtained by superimposing the output signal Vtp2 corresponding to the temperature-dependent electromotive force of the sensing element S2 on the reference potential Vref2.

The output signal Vtp1 is amplified by the differential amplifier 67 included in the signal processing circuit 60A to generate a gas detection signal Vgas1. The differential amplifier 67 compares the reference potential Vref2 supplied to the inversion input terminal (-) thereof with the level of (Vref2 + Vtp1) supplied to the non-inversion input terminal (+) thereof to generate the gas detection signal Vgas1 by amplifying the level difference (= Vtp1) therebetween.

The output signal Vtp2 is amplified by the differential amplifier 68 included in the signal processing circuit 60A to generate a gas detection signal Vgas2. The differential amplifier 68 compares the reference potential Vref2 supplied to the inversion input terminal (-) thereof with the level of (Vref2 + Vtp2) supplied to the non-inversion input terminal (+) thereof to generate the gas detection signal Vgas2 by amplifying the level difference (= Vtp2) therebetween.

A differential amplifier 69 compares the gas detection signal Vgas1 with the gas detection signal Vgas2 to generate an amplification signal Vamp0 corresponding to the amplified level difference (= Vgas1 – Vgas2) between the gas detection signal Vgas1 and the gas detection signal Vgas2. The amplification signal Vamp0 is supplied to the differential amplifier 63. The differential amplifier 63 compares the amplification signal Vamp0 with the reference potential Vref1 to generate an amplification signal Vamp1 corresponding to the amplified level difference (= Vamp0 – Vref1) between the amplification signal Vamp0 and the reference potential Vref1.

Even with such a circuit configuration, in a case where the heater is heated during measurement, when the CO₂ gas concentration in the measurement atmosphere increases, the thermal conductivities of the spaces between the heater MH3 and the sensing elements S1 and S2 decrease, so that the temperature difference between the sensing elements S1 and S2 decreases. As a result, the amplification signal Vamp0 changes (decreases). The amplification signal Vamp0 is compared with the reference potential Vref1 by the differential amplifier 63, and the amplification signal Vamp1 corresponding to the amplified level difference (= Vamp0 – Vref1) between the amplification signal Vamp0 and the reference potential Vref1 is generated. The amplification signal Vamp1 is supplied to the control circuit 66 through the AD converter 64. As a result, the output signal Vout indicating the CO₂ gas concentration in the measurement atmosphere is generated.

As exemplified by the sensor device 200B according to the fifth modification, the sensor device may output a signal (output signal Vout) based on the difference between the output (= Vtp1) of the sensing element S1 and the output (= Vtp2) of the sensing element S2. Further, although the sensor device 200B has the adhesive members 41 and 42 in the regions A1 and A2 of the sensor chip 20, respectively, the adhesive members 41 and 42 may be omitted, as in the sensor device 200 illustrated in FIG. 8.

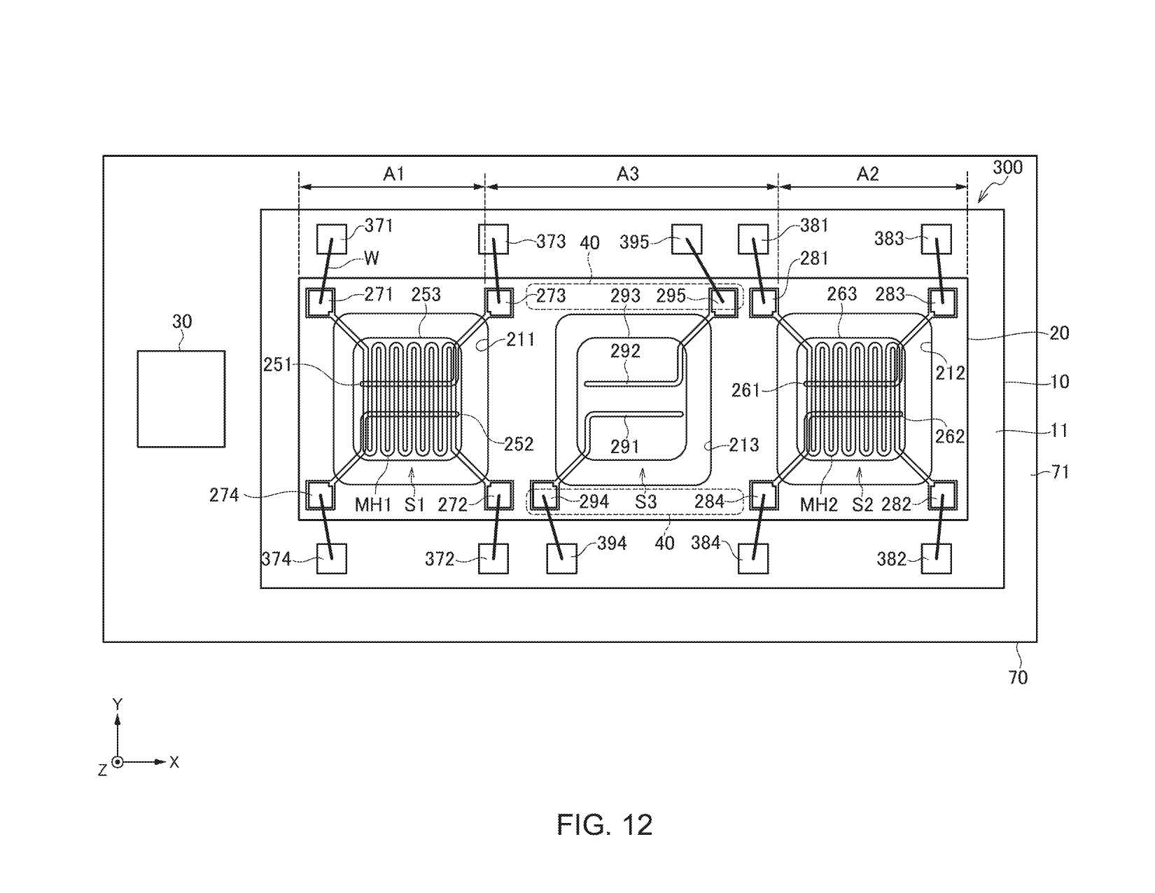

FIG. 12 is a schematic plan view illustrating the configuration of a sensor device 300 according to a third embodiment of the technology described herein.

As illustrated in FIG. 12, the sensor device 300 according to the third embodiment differs from the sensor device 100 according to the first embodiment in that the sensor chip 20 has a cavity 213 formed therein, and a sensing element S3 is disposed in the cavity 213. The sensing element S3 is a temperature sensor, for example, for measuring ambient temperature. The output signal of the sensing element S3 as a temperature sensor is supplied to the signal processing circuit 60 illustrated in FIG. 3, for example, for use in adjustment of the heater voltages Vmh1 and Vmh2 in accordance with ambient temperature.

The sensing element S3 is composed of a pair of thermistor electrodes 291, 292 and a thermistor resistor 293 contacting the pair of thermistor electrodes 291, 292. The thermistor electrode 291 is connected to a pad electrode 294, and the thermistor electrode 292 is connected to a pad electrode 295. The pad electrode 294 is connected to a pad electrode 394 provided on the main surface 11 of the sensor substrate 10 through the bonding wire W, and the pad electrode 295 is connected to a pad electrode 395 provided on the main surface 11 of the sensor substrate 10 through the bonding wire W.

The sensing element S3 is interposed between the sensing elements S1 and S2 in the X-direction. In the present embodiment, the adhesive member 40 is disposed at two separate locations in the region A3. The adhesive members 40, disposed in two separate locations in the region A3, partially sandwich the sensing element S3 in the Y-direction in a plan view as seen from the Z-direction. The adhesive members 40, disposed in two separate locations in the region A3, may entirely sandwich the sensing element S3 in the Y-direction in a plan view as seen from the Z-direction. This increases the distance between the adhesive member 40 and sensing elements S1 and S2, thereby reducing heat conduction from the electronic component 30 through the sensor substrate 10 to the sensing elements S1 and S2.

As exemplified by the sensor device 300 according to the third embodiment, the sensor chip 20 may additionally include the sensing element S3. Further, the sensing elements S1 to S3 may be arranged in a line along the X-direction.

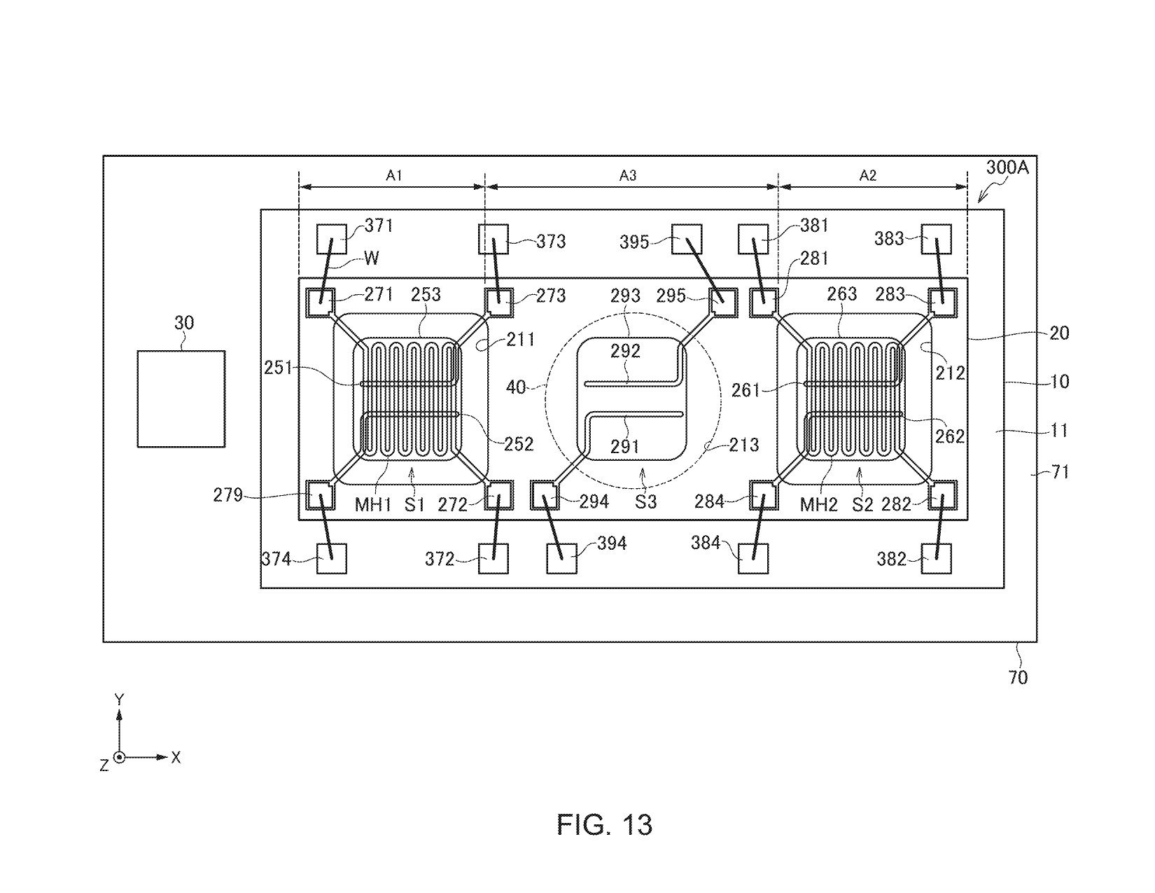

FIG. 13 is a schematic plan view illustrating the configuration of a sensor device 300A according to a sixth modification.

The sensor device 300A according to the sixth modification illustrated in FIG. 13 differs from the sensor device 300 illustrated in FIG. 12 in that the cavity 213 is omitted, and the adhesive member 40 is provided at a position overlapping the sensing element S3. In the sixth modification illustrated in FIG. 13, the cavity 213 is not formed below the sensing element S3, thereby enlarging the range in which the adhesive member 40 can be arranged.

In the sensor device 300 according to the third embodiment and the sensor device 300A according to the sixth modification, the influence of heat conduction from the electronic component 30 is greater on the sensing element S3 than on the sensing elements S1 and S2. However, as described using FIG. 3, since the sensing elements S1 and S2 constitute a half-bridge circuit, when a large difference occurs between the amount of heat conducted to the sensing element S1 through the sensor substrate 10 and the adhesive member 40 and that conducted to the sensing element S2 through the sensor substrate 10 and the adhesive member 40, it results in a large measurement error; on the other hand, the sensing element S3 as a temperature sensor does not constitute a bridge circuit with another sensing element, so that the measurement error caused by the heat conducted to the sensing element S3 through the sensor substrate 10 and the adhesive member 40 is smaller than the measurement error caused by heat conducted to the sensing elements S1 and S2.

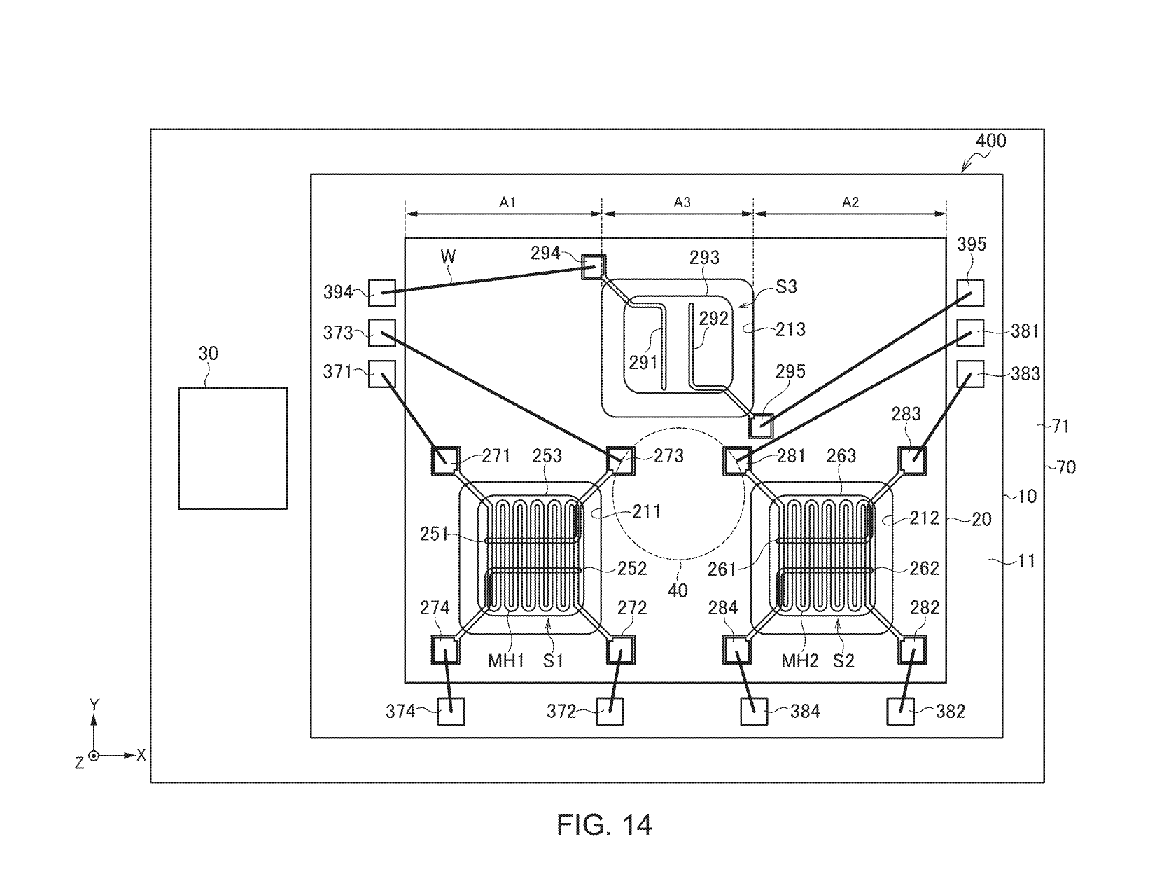

FIG. 14 is a schematic plan view illustrating the configuration of a sensor device 400 according to a fourth embodiment of the technology described herein.

As illustrated in FIG. 14, the sensor device 400 according to the fourth embodiment differs from the sensor device 300 according to the third embodiment in the position of the sensing element S3.

In the present embodiment, the sensing elements S1 and S2 are arranged in the X-direction, while the position of the sensing element S3 in the Y-direction differs from the positions of the sensing elements S1 and S2 in the Y-direction. As a result, a triangular region having the sensing elements S1, S2, and S3 as its vertices is formed. In the example illustrated in FIG. 14, the adhesive member 40 is disposed in the region A3 so as to overlap the triangular region. Although the sensing element S3 is disposed in the region A3 of the sensor chip 20 in the example illustrated in FIG. 14, the sensing element S3 may be disposed, wholly or partially, in the region A1 or A2 of the sensor chip 20.

Although some elements, such as the shapes of the sensor substrate 10 and the sensor chip 20 and the positions of the pad electrodes provided on the main surface 11 of the sensor substrate 10 differ from those of the sensor device 300 according to the third embodiment, the basic configuration is the same as that of the sensor device 300 according to the third embodiment, so the same reference numerals are given to the same elements, and overlapping description will be omitted.

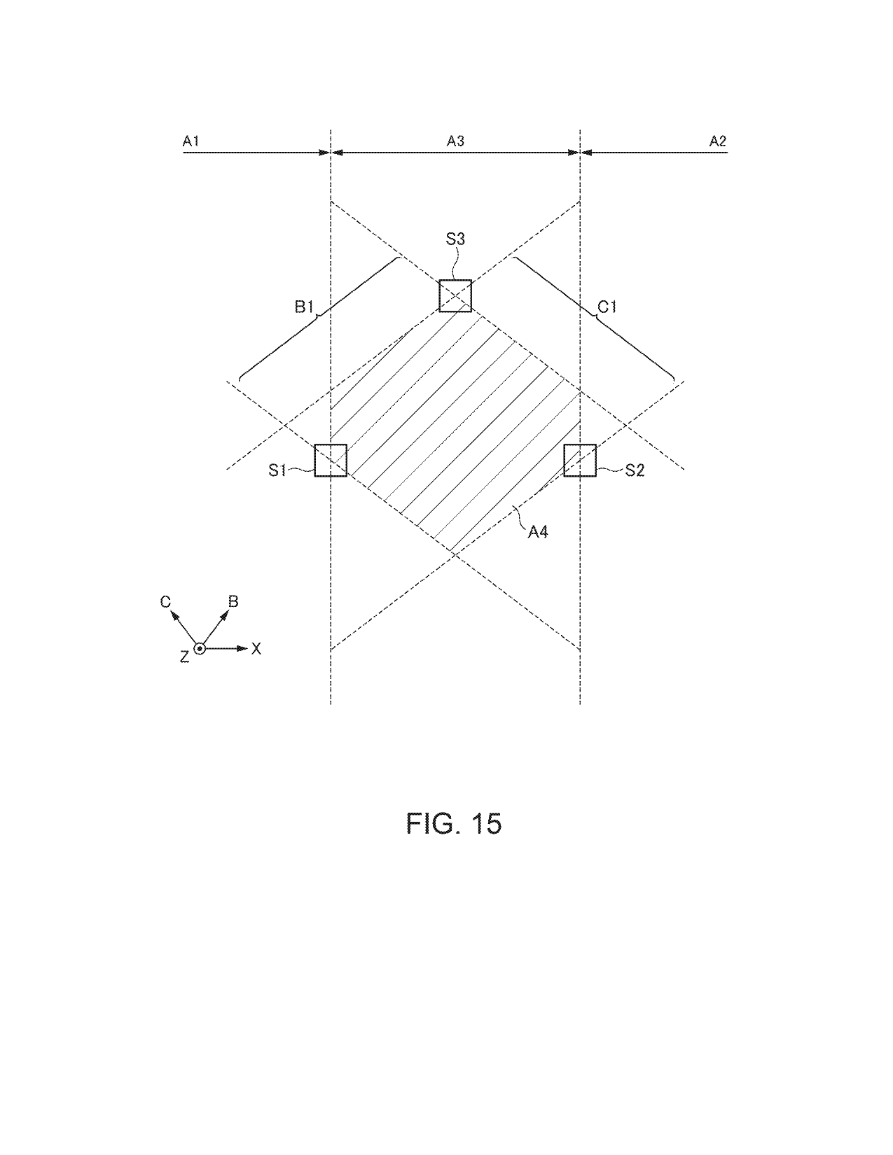

FIG. 15 is a schematic view for explaining the positional relationship between the sensing elements S1 to S3 and the adhesive member 40.

As illustrated in FIG. 15, when the arrangement direction of the sensing elements S1 and S3 is taken as “B-direction”, and the arrangement direction of the sensing elements S2 and S3 is taken as “C-direction”, the area between the position of the sensing element S1 in the B-direction and the position of the sensing element S3 in the B-direction in a plan view as seen from the Z-direction is defined as “region B1”, and the area between the position of the sensing element S2 in the C-direction and the position of the sensing element S3 in the C-direction in a plan view as seen from the Z-direction is defined as “region C1”. The X-, B-, and C-directions are mutually different directions. The adhesive member 40 is disposed within a region A4, which corresponds to an overlapping region of the regions B1 and C1 and is included in the region A3. In other words, the B-direction position of the adhesive member 40 disposed in the region A3 lies between the B-direction position of the sensing element S1 and the B-direction position of the sensing element S3, and the C-direction position of the adhesive member 40 disposed in the region A3 lies between the C-direction position of the sensing element S2 and the C-direction position of the sensing element S3. With this configuration, the difference between the amounts of heat conducted from the sensor substrate 10 to the sensing elements S1 to S3 through the adhesive member 40 is reduced. That is, the difference in the influence of heat conduction from the electronic component 30 between the sensing elements S1 to S3 is reduced.

In particular, when the adhesive member 40 is disposed so as to overlap the triangular region having the sensing elements S1, S2, and S3 as its vertices, the difference between the amounts of heat conducted from the sensor substrate 10 to the sensing elements S1 to S3 through the adhesive member 40 is further reduced. That is, the difference in the influence of heat conduction from the electronic component 30 between the sensing elements S1 to S3 is further reduced.

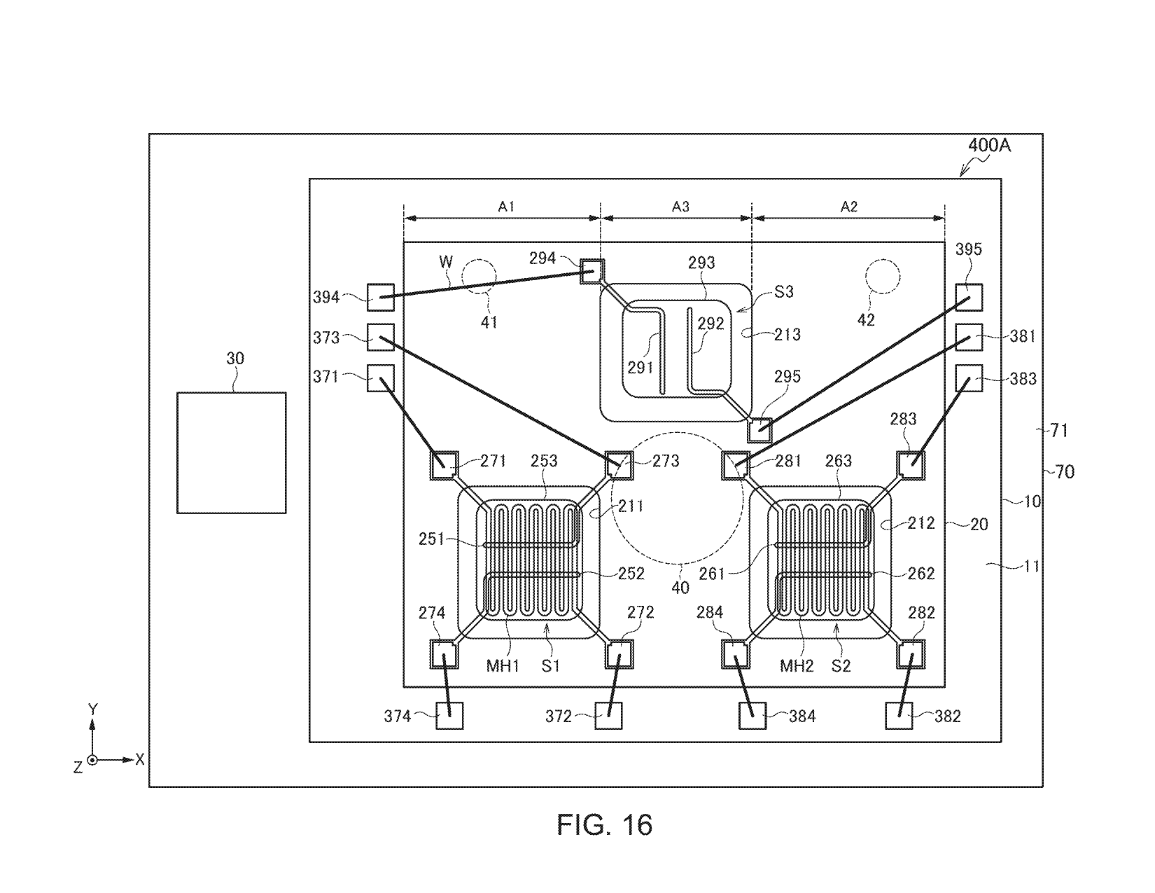

FIG. 16 is a schematic plan view illustrating the configuration of a sensor device 400A according to a seventh modification.

The sensor device 400A according to the seventh modification illustrated in FIG. 16 differs from the sensor device 400 illustrated in FIG. 14 in that adhesive members 41 and 42 are additionally provided in the regions A1 and A2 of the sensor chip 20, respectively. The adhesive members 41 and 42 each have a smaller volume than the adhesive member 40 provided in the region A3, and the adhesive member is disposed predominantly in the region A3 of the sensor chip. Thus, the heat conduction from the sensor substrate 10 to the sensor chip 20 is greater through the adhesive members 40 than through the adhesive members 41 and 42. In the sensor device 400A, the adhesive member is disposed predominantly in the region A4 of the sensor chip 20. Further, the B-direction position of the adhesive member 40 disposed in the region A3 lies between the B-direction position of the sensing element S1 and the B-direction position of the sensing element S3, and the C-direction position of the adhesive member 40 disposed in the region A3 lies between the C-direction position of the sensing element S2 and the C-direction position of the sensing element S3. With this configuration, the difference between the amounts of heat conducted from the sensor substrate 10 to the sensing elements S1 to S3 through the adhesive member 40 is reduced. That is, the difference in the influence of heat conduction from the electronic component 30 between the sensing elements S1 to S3 is reduced. Further, in the sensor device 400A, the adhesive member is disposed predominantly in the triangular region having the sensing elements S1, S2, and S3 as its vertices. Thus, the difference between the amounts of heat conducted from the sensor substrate 10 to the sensing elements S1 to S3 through the adhesive member 40 is further reduced. That is, the difference in the influence of heat conduction from the electronic component 30 between the sensing elements S1 to S3 is further reduced.

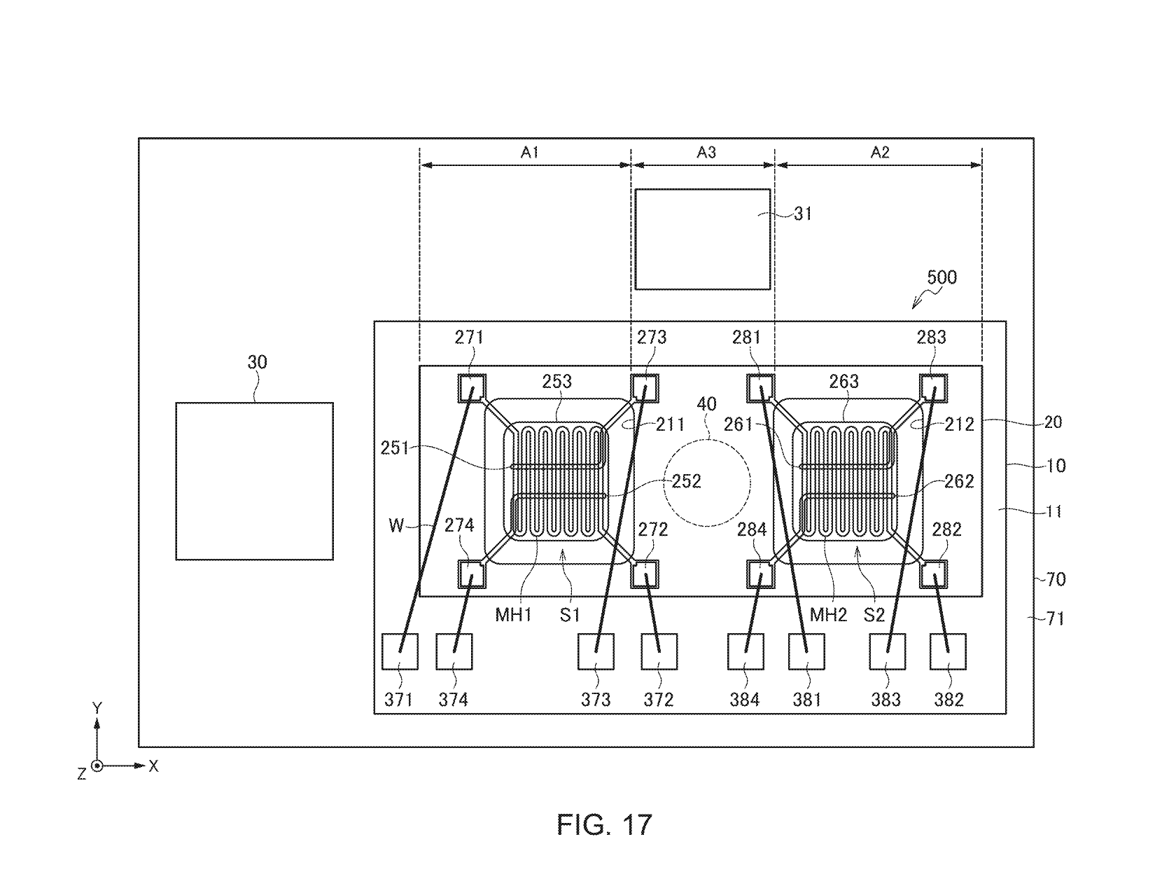

FIG. 17 is a schematic plan view illustrating the configuration of a sensor device 500 according to a fifth embodiment of the technology described herein.

As illustrated in FIG. 17, the sensor device 500 according to the fifth embodiment differs from the sensor device 100 according to the first embodiment in the following respects: another electronic component 31 is mounted on the mounting surface 71 of the product substrate 70; and, in a plan view as seen from the Z-direction, the pad electrodes 371 to 374 and 381 to 384 are arranged on the same side of the sensor chip 20, in a line along the X-direction. The electronic component 31 is a heating member that generates heat during its operation. In the present embodiment, in a plan view as seen from the Z-direction, the electronic component 31 is disposed on the on the positive side in the Y-direction with respect to the sensor device 500, and the pad electrodes 371 to 374 and 381 to 384 are disposed on the negative side in the Y-direction with respect to the sensor chip 20.

Although some elements, such as the shape of the sensor substrate 10 differ from those of the sensor device 100 according to the first embodiment, the basic configuration is the same as that of the sensor device 100 according to the first embodiment, so the same reference numerals are given to the same elements, and overlapping description will be omitted.

As exemplified by the sensor device 500 according to the fifth embodiment, when the pad electrodes 371 to 374 and 381 to 384 are disposed on one side with respect to the sensor chip 20 in a plan view as seen from the Z-direction, heat conduction from the electronic component 31, which is located on the other side with respect to the sensor chip 20, to the sensor substrate 10 is less likely to be conducted through the bonding wires W. Although, in the example illustrated in FIG. 17, the pad electrodes 371 to 374 and 381 to 384 are arranged in a line, they may be arranged in a plurality of lines on the same side with respect to the sensor chip 20 in a plan view as seen from the Z-direction.

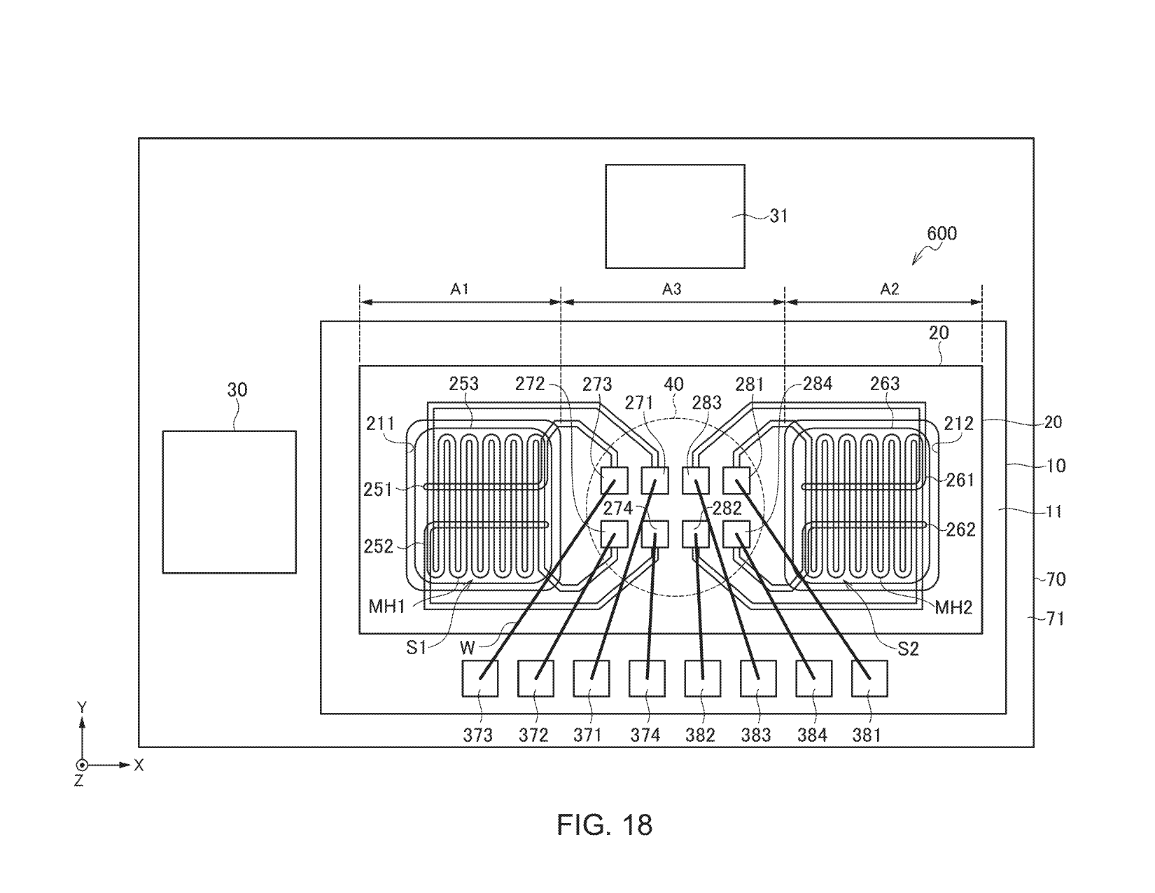

FIG. 18 is a schematic plan view illustrating the configuration of a sensor device 600 according to a sixth embodiment of the technology described herein.

As illustrated in FIG. 18, the sensor device 600 according to the sixth embodiment differs from the sensor device 500 according to the fifth embodiment in that the pad electrodes 271 to 274 and 281 to 284 on the sensor chip 20 are disposed in the region A3. In the present embodiment, in a plan view as seen from the Z-direction, the pad electrodes 271 to 274 and281 to 284 are collectively disposed between the sensing elements S1 and S2.

Although some elements, such as the arrangement of the pad electrodes 371 to 374 and 381 to 384, differ from those of the sensor device 500 according to the fifth embodiment, the basic configuration is the same as that of the sensor device 500 according to the fifth embodiment, so the same reference numerals are given to the same elements, and overlapping description will be omitted.

As exemplified by the sensor device 600 according to the sixth embodiment, when the pad electrodes 271 to 274 and 281 to 284 are collectively disposed in the region A3 of the sensor chip 20, it is possible to reduce the difference between the amount of heat conducted to the sensing element S1 through the bonding wires W and the amount of heat conducted to the sensing element S2 through the bonding wires W, thereby making it possible to further reduce a measurement error caused by the heat generation of the electronic components 30 and 31.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, in the above embodiments, the sensing elements S1 and S2 have been described by way of example as elements using a thermistor resistor or as thermocouples; however, the present invention is not limited thereto, and other types of elements whose characteristics vary with temperature may also be employed. Further, in the above embodiments, the sensing elements S1 and S2 have been described by way of example as elements used for a heat conduction type gas sensor; however, the types of the sensing elements S1 and S2 are not limited as long as they are used for a sensor in which heat from an external heating member affects its measurement accuracy. Further, the type of a sensor for which the sensing elements S1 and S2 are used is not limited as long as it is a sensor in which heat from an external heating member affects its measurement accuracy.

Further, the sensor device according to the present disclosure need not be a sensor device used for a gas sensor as long as it measures a certain physical quantity based on the states of the sensing elements S1 and S2 and may be a sensor device configured to measure another physical quantity. Further, it is not essential that one of the sensing elements S1 and S2 is an element for detection and the other one is an element for reference, but both the sensing elements S1 and S2 may be elements for detection.

The technology according to the present disclosure includes the following configuration examples, but not limited thereto.

A sensor device according to an aspect of the present disclosure includes: a sensor substrate; a sensor chip mounted on the sensor substrate and having a first sensing element and a second sensing element; and an adhesive member provided between the sensor chip and the sensor substrate so as to fix the sensor chip onto the sensor substrate, wherein the sensor chip has a first region, a second region, and a third region interposed between the first region and the second region in a first direction, the first sensing element is disposed in the first region of the sensor chip, the second sensing element is disposed in the second region of the sensor chip, and the adhesive member is disposed predominantly in the third region of the sensor chip. With this configuration, the difference between the amount of heat conducted from the sensor substrate to the first sensing element and that conducted from the sensor substrate to the second sensing element is reduced. That is, the difference in the influence of heat conduction from a heating member between the first and second sensing elements is reduced.

In the above sensor device, the adhesive member may be disposed in the third region of the sensor chip without disposed in the first and second regions of the sensor chip. This further reduces the difference between the amount of heat conducted from the sensor substrate to the first sensing element and that conducted from the sensor substrate to the second sensing element.

In the above sensor device, in a plan view as seen from a thickness direction of the sensor substrate, at least a part of the adhesive member may be disposed in the third region interposed between the first sensing element and the second sensing element in the first direction. This makes it possible to reduce the size of the sensor chip in a second direction crossing the first direction.

In the above sensor device, in a plan view as seen from a thickness direction of the sensor substrate, at least a part of the adhesive member may be disposed in the third region outside a region interposed between the first sensing element and the second sensing element in the first direction. This makes it possible to increase the distance between the adhesive member and the first and second sensing elements.

In the above sensor device, in a plan view as seen from the thickness direction of the sensor substrate, the first sensing element and the second sensing element may be disposed in a single cavity. This makes it possible to reduce the size of the sensor chip.

In the above sensor device, in a plan view as seen from the thickness direction of the sensor substrate, at least parts of the adhesive members may be disposed in the third region so as to interpose the cavity in a second direction crossing the first direction. This makes it possible to increase the distance between the adhesive member and the first and second sensing elements.

In the above sensor device, the sensor chip may further include a third sensing element, and the third sensing element may be interposed between the first sensing element and the second sensing element in the first direction. This maintains a sufficient distance between the first and second sensing elements, making heat interference between the first and second sensing elements unlikely.

In the above sensor device, in a plan view as seen from a thickness direction of the sensor substrate, at least parts of the adhesive members may be disposed in the third region so as to interpose the third sensing element in a second direction crossing the first direction. This makes it possible to increase the distance between the adhesive member and the first and second sensing elements.

In the above sensor device, the sensor chip may further include a third sensing element, the first sensing element and the third sensing element may be arranged in a second direction, the second sensing element and the third sensing elements are arranged in a third direction, and, in a plan view as seen from a thickness direction of the sensor substrate, a position of the adhesive member disposed in the third region in the second direction may lie between a position of the first sensing element in the second direction and a position of the third sensing element in the second direction, and a position of the adhesive member disposed in the third region in the third direction may lie between a position of the second sensing element in the third direction and a position of the third sensing element in the third direction. With this configuration, the difference between the amounts of heat conducted from the sensor substrate, through the adhesive member, to the first to third sensing elements is reduced, thereby reducing the difference in the influence of heat conduction from a heating member between the first to third sensing elements.

In the above sensor device, the third sensing element may be disposed in the third region.

In the above sensor device, in a plan view as seen from the thickness direction of the sensor substrate, at least a part of the adhesive member may be disposed in the third region so as to overlap a triangular region having the first sensing element, the second sensing element, and third sensing element as vertices of the triangular region, and the adhesive member may be disposed predominantly in the triangular region. This further reduces the difference between the amounts of heat conducted from the sensor substrate to the first to third sensing elements.

The above sensor device may further include a plurality of bonding wires electrically connecting the sensor substrate and the sensor chip, the sensor chip may have a plurality of first pad electrodes, the sensor substrate may have a plurality of second pad electrodes, each of the bonding wires may connect corresponding one of the first pad electrodes and corresponding one of the second pad electrodes, and in a plan view as seen from a thickness direction of the sensor substrate, the second pad electrodes may be disposed on one side with respect to the sensor chip. This makes it possible to reduce the influence caused by the heat generation of a heating member mounted on the opposite side of the second pads with respect to the sensor chip.

The above sensor device may further include a plurality of bonding wires electrically connecting the sensor substrate and the sensor chip, the sensor chip may have a plurality of first pad electrodes, the sensor substrate may have a plurality of second pad electrodes, each of the bonding wires may connect corresponding one of the first pad electrodes and corresponding one of the second pad electrodes, and the first pad electrodes may be disposed in the third region. This reduces the difference between the amounts of heat conducted from the sensor substrate to the first and second sensing elements through the bonding wires.

What is claimed is:

1. A sensor device comprising:
a sensor substrate;
a sensor chip mounted on the sensor substrate and having a first sensing element and a second sensing element; and
an adhesive member provided between the sensor chip and the sensor substrate so as to fix the sensor chip onto the sensor substrate,
wherein the sensor chip has a first region, a second region, and a third region interposed between the first region and the second region in a first direction,
wherein the first sensing element is disposed in the first region of the sensor chip,
wherein the second sensing element is disposed in the second region of the sensor chip, and
wherein the adhesive member is disposed predominantly in the third region of the sensor chip.

2. The sensor device as claimed in claim 1, wherein the adhesive member is disposed in the third region of the sensor chip without disposed in the first and second regions of the sensor chip.

3. The sensor device as claimed in claim 1, wherein, in a plan view as seen from a thickness direction of the sensor substrate, at least a part of the adhesive member is disposed in the third region interposed between the first sensing element and the second sensing element in the first direction.

4. The sensor device as claimed in claim 1, wherein, in a plan view as seen from a thickness direction of the sensor substrate, at least a part of the adhesive member is disposed in the third region outside a region interposed between the first sensing element and the second sensing element in the first direction.

5. The sensor device as claimed in claim 4, wherein, in a plan view as seen from the thickness direction of the sensor substrate, the first sensing element and the second sensing element are disposed in a single cavity.

6. The sensor device as claimed in claim 5, wherein, in a plan view as seen from the thickness direction of the sensor substrate, at least parts of the adhesive members are disposed in the third region so as to interpose the cavity in a second direction crossing the first direction.

7. The sensor device as claimed in claim 1,
wherein the sensor chip further includes a third sensing element, and
wherein the third sensing element is interposed between the first sensing element and the second sensing element in the first direction.

8. The sensor device as claimed in claim 7, wherein, in a plan view as seen from a thickness direction of the sensor substrate, at least parts of the adhesive members are disposed in the third region so as to interpose the third sensing element in a second direction crossing the first direction.

9. The sensor device as claimed in claim 1,
wherein the sensor chip further includes a third sensing element,
wherein the first sensing element and the third sensing element are arranged in a second direction,
wherein the second sensing element and the third sensing elements are arranged in a third direction, and
wherein, in a plan view as seen from a thickness direction of the sensor substrate, a position of the adhesive member disposed in the third region in the second direction lies between a position of the first sensing element in the second direction and a position of the third sensing element in the second direction, and a position of the adhesive member disposed in the third region in the third direction lies between a position of the second sensing element in the third direction and a position of the third sensing element in the third direction.

10. The sensor device as claimed in claim 9, wherein the third sensing element is disposed in the third region.

11. The sensor device as claimed in claim 10,
wherein, in a plan view as seen from the thickness direction of the sensor substrate, at least a part of the adhesive member is disposed in the third region so as to overlap a triangular region having the first sensing element, the second sensing element, and the third sensing element as vertices of the triangular region, and
wherein the adhesive member is disposed predominantly in the triangular region.

12. The sensor device as claimed in claim 1, further comprising a plurality of bonding wires electrically connecting the sensor substrate and the sensor chip,
wherein the sensor chip has a plurality of first pad electrodes,
wherein the sensor substrate has a plurality of second pad electrodes,
wherein each of the bonding wires connects corresponding one of the first pad electrodes and corresponding one of the second pad electrodes, and
wherein, in a plan view as seen from a thickness direction of the sensor substrate, the second pad electrodes are disposed on one side with respect to the sensor chip.

13. The sensor device as claimed in claim 1, further comprising a plurality of bonding wires electrically connecting the sensor substrate and the sensor chip,
wherein the sensor chip has a plurality of first pad electrodes,
wherein the sensor substrate has a plurality of second pad electrodes,
wherein each of the bonding wires connects corresponding one of the first pad electrodes and corresponding one of the second pad electrodes, and
wherein the first pad electrodes are disposed in the third region.
